(19) United States
(12) Patent Application Publication
(10) Pub. No.: US 2026/0234534 A1
(43) Pub. Date: Aug. 13, 2026

(54) MEMBRANE MECHANOPORATION DEVICES AND METHODS FOR TRANSFECTING CELLS

(71) Applicant: Portal Biotechnologies, Inc., Arlington, MA (US)

(72) Inventors: Rachel Eris CONOVER, Durham, NC (US); Cole Jeffrey CONSTANTINEAU, Cambridge, MA (US); Jonathan Brian GILBERT, Arlington, MA (US); Collin Tyler MASON, Somerville, MA (US); Armon Reza SHAREI, Arlington, MA (US); Alec John BARCLAY, Winchester, MA (US); Zhihui SONG, Philadelphia, PA (US); Andrew Francis LAROCQUE, Hamden, CT (US)

(21) Appl. No.: 19/160,316

(22) PCT Filed: Feb. 27, 2024
(86) PCT No.: PCT/US2024/017527

§ 371 Date: Aug. 27, 2025

Related U.S. Application Data
(60) Provisional application No. 63/449,013, filed on Feb. 28, 2023.

(51) Int. Cl. C12M 1/42; C12M 1/00; C12M 1/34; C12N 15/87

(52) U.S. Cl. CPC C12M 35/04; C12M 29/04; C12M 41/40; C12N 15/87

(57) ABSTRACT

The present disclosure provides systems, devices, and methods for mechanoporation of cells to facilitate delivery of a pay load into the cells. The mechanoporation systems described herein can simplify cell processing systems by providing a tubing set that is disposable, compatible with existing pumps and other cell processing equipment, and operable at lower pressures constituted by these existing pump systems. The tubing set can mix a cell suspension received from upstream cell processing equipment with a payload optionally contained within the tubing set and can perturb the membranes of the cells by passing the cells through a cell perturbation filter of the tubing set.

### CROSS-REFERENCE TO RELATED APPLICATIONS

This application claims priority to and the benefit of U.S. Provisional Patent Application No. 63/449,013, filed Feb. 28, 2023, the entire contents of which are hereby incorporated by reference.

### FIELD

This disclosure relates generally to intracellular delivery of a payload, and more specifically to systems comprising tubing sets compatible with existing pump systems that perturb cell membranes to allow delivery of a payload into the cell.

### BACKGROUND

Intracellular delivery of macromolecules such as nucleic acids, peptides and proteins is a challenge in research and therapeutic applications. The delivery of the aforementioned materials into cells can be controlled by rapidly mechanically deforming the cell to produce transient cell membrane disruptions that allow the macromolecules to enter the cell cytosol. Known techniques of mechanical deformation of cells include the use of constricting microfluidic channels or porous surfaces (e.g., filters) that cause perturbations in the cell membranes. These microfluidic channels or filters are embodied in cartridges that require specialized pump systems or other pressure mechanisms (e.g., gas cylinders) to drive cells through the channels or pores of the filters.

Cell processing systems including filtration and elutriation systems are used extensively in labs and medical facilities to prepare cell samples for various therapies. For example, cell processing systems are used for cell isolation, suspension, dilution, incubation, and/or cell washing. One or more of these functionalities can require an individual system (i.e., equipment) for execution. Systems which attempt to incorporate multiple of the aforementioned functionalities can be unwieldy and costly.

### SUMMARY

Described herein are systems, devices, and methods for delivering a payload into cells. As mentioned above, known techniques for delivering payloads into cells include mechanically deforming the cell membrane using microfluidic channels and/or pores. For example, cells may be passed through constrictions comprising a smaller diameter than the cell, which results in the formation of disruptions or holes in the cell membrane. The constrictions may comprise one or more microfluidic channels and/or porous surfaces (e.g., filters) integrated to fluidic cartridges for cell therapy applications. These existing cartridge systems use specially designed pumping and/or pressure-driving equipment (e.g., gas cylinders) to drive cells through the cartridge and cause mechanoporation of the cells, thus increasing the amount of bulky equipment in lab or clinical areas already scarce for space. The safety and portability of such existing systems can be complicated based on high pressure requirements of the pressure-driving equipment. Furthermore, existing cartridge systems may require separate, standalone modules and/or processes to perform individual tasks in cell processing (e.g., mixing, which may be performed manually by technicians). Adding to the number of devices, systems, and processes increases the number of technicians needed to set-up and operate the various devices. Additional technicians and equipment may cause an increase in the processing time of cells as well as an increase in cost.

Accordingly, improved systems for mechanoporation of cells are needed. The disclosed mechanoporation systems, devices, and methods may provide a disposable tubing set comprising cartridge(s) for use in clinical and lab settings that can mix a payload and a cell suspension and perturb the membranes of cells in the cell suspension to allow the payload to be delivered to the cells. The tubing set may be compatible with various existing pumps (e.g., peristaltic pumps, syringe pumps, etc.), filtration systems, and elutriation systems that can provide the cell suspension to the tubing set for mechanoporation. Thus, cell processing can be simplified, and scarce clinical and lab space can be preserved by minimizing excess equipment. Reducing the complexity of and amount of equipment required for cell processing may also reduce barriers of entry to cell therapies by reducing costs associated with large equipment and complex systems. The pressure requirements of the tubing set may be lower than that of existing cartridge systems, thereby improving portability, safety, and compatibility of the system. For example, once connected to existing pumping systems, the tubing set may adapt to and operate based on the system pressure provided by the existing pumps. The ability of the tubing set to adapt to the pressure provided by an external pump and/or filtration system can allow the tubing set to maintain the cell speed necessary to facilitate perturbation of the cell membrane as the cell suspension passes through the cell perturbation filters. Moreover, the closed system comprising existing pumping equipment and the tubing set described herein may be operable outside of a clean-room (e.g., in a point-of-care setting) and may integrate multiple cell processing steps in a single consumable tubing set, necessitating fewer technicians.

The tubing sets described herein may enable efficacious mechanoporation of large quantities of cells while maintaining high cell viability and cell retention. For example, the tubing set (i.e., the cell perturbation filter(s) within the tubing set) may achieve a delivery efficiency, quantified by the delivery of the payload into the cells, of at least 60%. The tubing set may achieve this delivery efficiency while maintaining the cell viability above about 60%. Moreover, the tubing set can be capable of processing millions of cells (e.g., greater than 200 million cells) whilst maintaining the delivery efficiency, cell viability, and cell retention of the sample. The tubing set may process this large quantity of cells in one pass without failure or clogging the cell perturbation filter(s) within the tubing set. The mechanoporation system may achieve high cell viability and delivery at flow rates through the system of at least 0.5 mL/min per mm² porous surface area of the cell perturbation filter. Accordingly, the tubing sets described herein achieve a balance of high cell throughput, delivery efficiency, and cell viability heretofore unachievable by existing mechanoporation systems.

In some embodiments, a tubing set for delivering a payload into cells is provided, the tubing set comprising: a cell source tubing portion configured to fluidly connect to a cell source comprising a cell suspension; a mixer fluidly connected to the cell source tubing portion and configured to mix a payload and the cell suspension to produce a mixture; a delivery portion comprising one or more cell perturbation filters fluidly connected to the mixer to receive the mixture and configured to perturb membranes of cells of the mixture as the mixture passes through the one or more cell perturbation filters; and a pump connection portion configured to connect to one or more pumps that are configured to move the cell suspension through the cell source tubing portion and into the mixer, to move the mixture from the mixer through the one or more cell perturbation filters, and to move the perturbed cell mixture into one or more collection reservoirs configured to collect the perturbed cell mixture.

In some embodiments, a tubing set for delivering a payload into cells is provided, the tubing set comprising: a cell source tubing portion configured to fluidly connect to a cell source comprising a cell suspension; a delivery portion comprising one or more cell perturbation filters fluidly connected to the cell source tubing portion to receive the cell suspension and configured to perturb membranes of cells of the cell suspension as the cell suspension passes through the one or more cell perturbation filters; a mixer fluidly connected to the delivery portion and configured to mix a payload and a perturbed cell suspension to produce a perturbed cell mixture; and a pump connection portion configured to connect to one or more pumps that are configured to move the cell suspension through the cell source tubing portion and through the one or more cell perturbation filters, to move the perturbed cell suspension from the delivery portion and into the mixer, and to move the perturbed cell mixture into one or more collection reservoirs configured to collect the perturbed cell mixture.

In some embodiments, a mechanoporation system for delivering a payload into cells is provided, the system comprising: a payload source comprising the payload; a cell source comprising a cell suspension; a tubing set comprising: a mixer fluidly connected to the payload source and to the cell source and configured to mix the payload and the cell suspension to produce a mixture; and one or more cell perturbation filters fluidly connected to the mixer to receive the mixture and configured to perturb membranes of cells of the mixture as the mixture passes through the one or more cell perturbation filters; one or more collection reservoirs fluidly connected to the one or more cell perturbation filters to collect the perturbed cell mixture; and one or more pumps connected to the tubing set and configured to move the payload and the cell suspension into the mixer, to move the mixture from the mixer through the one or more cell perturbation filters, and to move the perturbed cell mixture into the one or more collection reservoirs.

In some embodiments, a method for delivering a payload into cells is provided, the method comprising: mixing, by a mixer fluidly connected to a payload source comprising a payload and to a cell source comprising a cell suspension, the payload and the cell suspension to produce a mixture; perturbing, by one or more cell perturbation filters fluidly connected to the mixer to receive the mixture, membranes of cells of the mixture as the mixture passes through the one or more cell perturbation filters; and collecting, by one or more collection reservoirs fluidly connected to the one or more cell perturbation filters, the perturbed cell mixture, wherein one or more pumps are configured to move the payload and the cell suspension into the mixer, to move the mixture from the mixer through the one or more cell perturbation filters, and to move the perturbed cell mixture into the one or more collection reservoirs.

### BRIEF DESCRIPTION OF THE FIGURES

The present application can be best understood by reference to the following description taken in conjunction with the accompanying figures included in the specification.

FIGS. 1A-1H illustrate mechanoporation system diagrams, in accordance with various embodiments. FIG. 1A illustrates a mechanoporation system in which a cell suspension from the cell source is passed through the payload source and into the tubing kit, in accordance with various embodiments. FIG. 1B illustrates a mechanoporation system in which the payload and the cell suspension is mixed by a mixer in the tubing kit, in accordance with various embodiments. FIG. 1C illustrates a mechanoporation system in which the tubing kit includes a mixture pressure regulator downstream of the mixer, in accordance with various embodiments. FIG. 1D illustrates a mechanoporation system in which each of the cell suspension and the payload separately pass through a pump prior to entering the tubing kit, in accordance with various embodiments. FIG. 1E illustrates a mechanoporation system in which the tubing kit includes a cell strainer, pressure regulator, pulsation damper, and cell perturbation filter, in accordance with various embodiments. FIG. 1F illustrates a mechanoporation system in which the tubing kit includes a plurality of cell mechanoporation filters arranged in parallel, in accordance with various embodiments. FIG. 1G illustrates a mechanoporation system in which the tubing kit includes a pulsation damper fluidly connected to a cell perturbation filter, in accordance with various embodiments. FIG. 1H illustrates a mechanoporation system in which the tubing kit includes a pulsation damper fluidly connected to a plurality of cell perturbation filters, in accordance with various embodiments.

FIGS. 2A-2J illustrate block diagrams of tubing set arrangements, in accordance with various embodiments. FIG. 2A illustrates a tubing set that includes a housing containing a mixer and a cell perturbation filter, in accordance with various embodiments. FIG. 2B illustrates a tubing set that includes a housing containing a mixer, cell perturbation filter, and a collection reservoir, in accordance with various embodiments. FIG. 2C illustrates a tubing set that includes a housing containing a payload reservoir, mixer, and cell perturbation filter, in accordance with various embodiments. FIG. 2D illustrates a tubing set that does not include separate payload source tubing, in accordance with various embodiments. FIG. 2E illustrates a tubing set that includes several cell perturbation filters arranged in parallel, in accordance with various embodiments. FIG. 2F illustrates a tubing set that includes several collection reservoirs configured to collect perturbed cell mixtures from several cell perturbation filters that are arranged in parallel, in accordance with various embodiments.

FIG. 2G illustrates a tubing set that includes a mixture tubing portion fluidly connected to a housing containing a cell strainer, pressure regulator, pulsation damper, and a cell perturbation filter, in accordance with some embodiments. FIG. 2H illustrates a tubing set that includes a mixture tubing portion fluidly connected to a housing containing a cell strainer, pressure regulator, pulsation damper, and a plurality of cell perturbation filters, in accordance with some embodiments. FIG. 2I illustrates a tubing set that includes a housing containing a pulsation damper and a cell perturbation filter, in accordance with various embodiments. FIG. 2J illustrates a tubing set that includes a housing containing a pulsation damper and a plurality of cell perturbation filters, in accordance with various embodiments.

FIG. 3 illustrates an exemplary mixer apparatus of a mechanoporation system, in accordance with various embodiments.

FIG. 4A illustrates an exemplary tubing set diagram that receives a cell mixture from an upstream cell processing system, in accordance with various embodiments. FIG. 4B illustrates cell viability results from testing the tubing set illustrated in FIG. 4A, in accordance with various embodiments. FIG. 4C illustrates delivery efficiency results from testing the tubing set illustrated in FIG. 4A, in accordance with various embodiments.

FIG. 5A illustrates another exemplary tubing set diagram that receives a cell mixture from an upstream cell processing system, in accordance with various embodiments.

FIG. 5B illustrates cell viability results from testing the tubing set illustrated in FIG. 5A, in accordance with various embodiments. FIG. 5C illustrates delivery efficiency results from testing the tubing set illustrated in FIG. 5A, in accordance with various embodiments.

FIG. 6A illustrates another exemplary tubing set diagram that receives a cell mixture from an upstream cell processing system, in accordance with various embodiments.

FIG. 6B illustrates cell viability results from testing the tubing set illustrated in FIG. 6A, in accordance with various embodiments. FIG. 6C illustrates delivery efficiency results from testing the tubing set illustrated in FIG. 6A, in accordance with various embodiments.

FIG. 7A illustrates another exemplary tubing set diagram that receives a cell mixture from an upstream cell processing system, in accordance with various embodiments. FIG. 7B illustrates cell viability results from testing the tubing set illustrated in FIG. 7A, in accordance with various embodiments. FIG. 7C illustrates delivery efficiency results from testing the tubing set illustrated in FIG. 7A, in accordance with various embodiments.

FIG. 8A illustrates another exemplary tubing set diagram that receives a cell mixture from an upstream cell processing system, in accordance with various embodiments. FIG. 8B illustrates cell viability results from testing the tubing set illustrated in FIG. 8A, in accordance with various embodiments. FIG. 8C illustrates delivery efficiency results from testing the tubing set illustrated in FIG. 8A, in accordance with various embodiments.

FIG. 9A illustrates another exemplary tubing set diagram that receives a cell mixture from an upstream cell processing system, in accordance with various embodiments. FIG. 9B illustrates an exemplary cell perturbation filter design used in the tubing set illustrated in FIG. 9A. FIG. 9C illustrates cell viability results from testing the tubing set illustrated in FIG. 9A, in accordance with various embodiments. FIG. 9D illustrates delivery efficiency results from testing the tubing set illustrated in FIG. 9A, in accordance with various embodiments.

FIGS. 10A-10D illustrate results from testing another exemplary tubing set that receives a cell mixture from an upstream cell processing system, in accordance with some embodiments. FIG. 10A illustrates cell retention results from testing the exemplary tubing set, in accordance with some embodiments. FIG. 10B illustrates cell viability results from testing the exemplary tubing set, in accordance with some embodiments. FIG. 10C illustrates delivery efficiency results from testing the exemplary tubing set, in accordance with some embodiments. FIG. 10D illustrates the presence of the payload in the cells passed through the exemplary tubing set, in accordance with some embodiments.

### DETAILED DESCRIPTION

In some aspects, systems, devices, and methods for delivering a payload to cells are described herein. As mentioned above, existing devices and systems for mechanically deforming cells to cause perturbation of the cell membranes use specialized equipment designed for pumping and mixing the materials prior to mechanoporation of the cells. The additional equipment adds to the number of devices and systems crowding already scarce clinical and lab space and may utilize additional technicians to operate said devices. The mechanoporation systems, devices, and methods of use thereof that are described herein can simplify cell processing systems by providing a tubing set for mechanoporation that reduces the complexity of the pumping device and leverages existing equipment, thus reducing any barriers to entry in cell therapies.

In some embodiments, the tubing set described herein may be disposable and attachable to various existing pumps, such as those integrated within cell washing systems, filtration systems, elutriation systems, isolation systems, and automated end-to-end cell therapy production systems, peristaltic pumps, and/or syringe pumps. These various pumps and systems may each have unique pressure and/or flow regimes, and the tubing set may be configured to adapt to each of the unique system parameters to facilitate mixing of a payload and cell suspension as well as mechanoporation of the cells to deliver a payload into the cells. The tubing set described herein may be configured to at least mix the cell suspension and the payload to produce a mixture and pass the mixture through one or more filters to perturb the cells. Each of the aforementioned requirements of the tubing set may be executed within a single, simple cartridge (hereinafter referred to as a housing) of the tubing set. The tubing set may be configured to receive and/or contain one or more of the cell suspension and/or payload. The tubing set may be configured to collect and/or provide the perturbed cell mixture for downstream cell therapy applications.

The tubing sets described herein may enable efficacious mechanoporation of large quantities of cells while maintaining high cell viability and cell retention. For example, the tubing set (i.e., the cell perturbation filter(s) within the tubing set) may achieve a delivery efficiency of at least 60%, which can be quantified by the delivery of the payload into the cells. The tubing set may achieve this delivery efficiency while maintaining the cell viability above about 60%. Moreover, the tubing set can be capable of processing a quantity of cells on the magnitude of millions of cells (e.g., greater than 200 million cells) whilst maintaining the delivery efficiency, cell viability, and cell retention of the sample. The tubing set may process this large quantity of cells efficiently in one pass without failure or clogging the cell perturbation filter(s) within the tubing set. The mechanoporation system may achieve high cell viability and delivery efficiency at flow rates of at least 0.5 mL/min per mm² porous surface area of the cell perturbation filter.

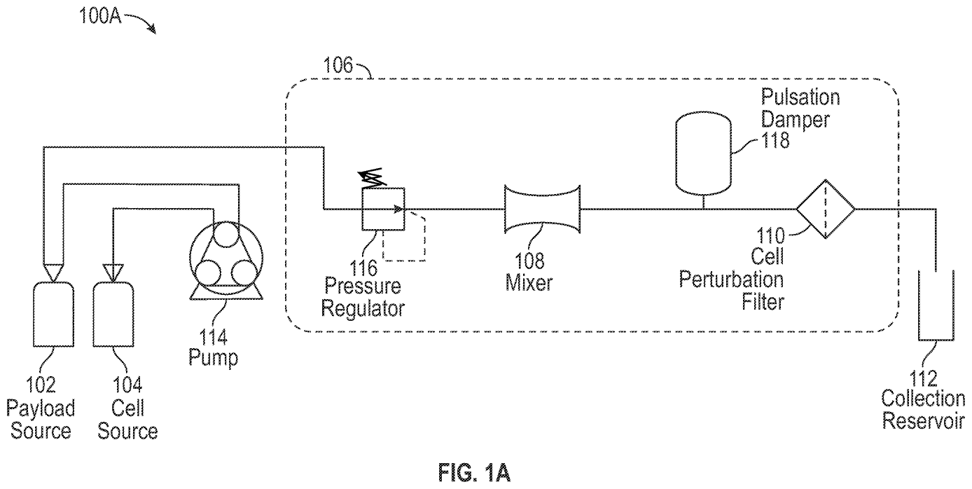

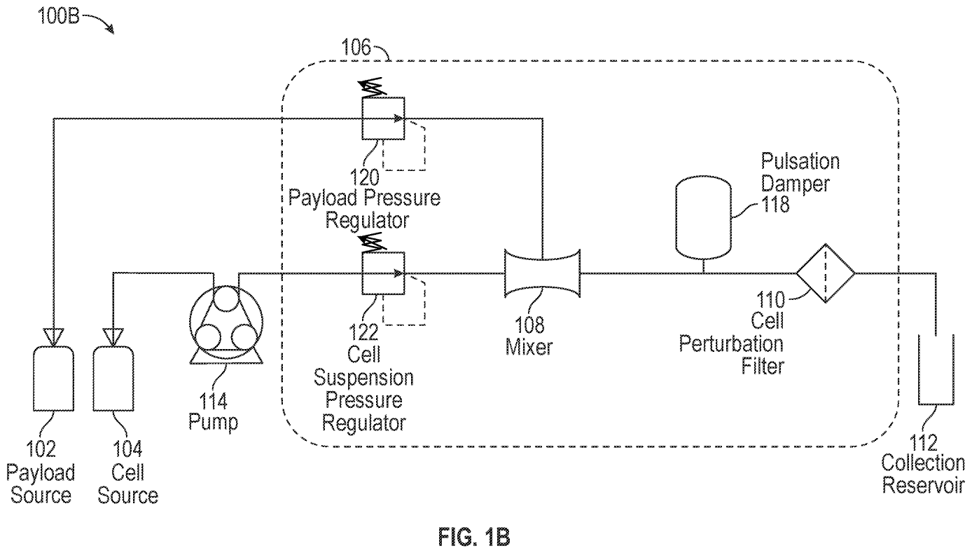

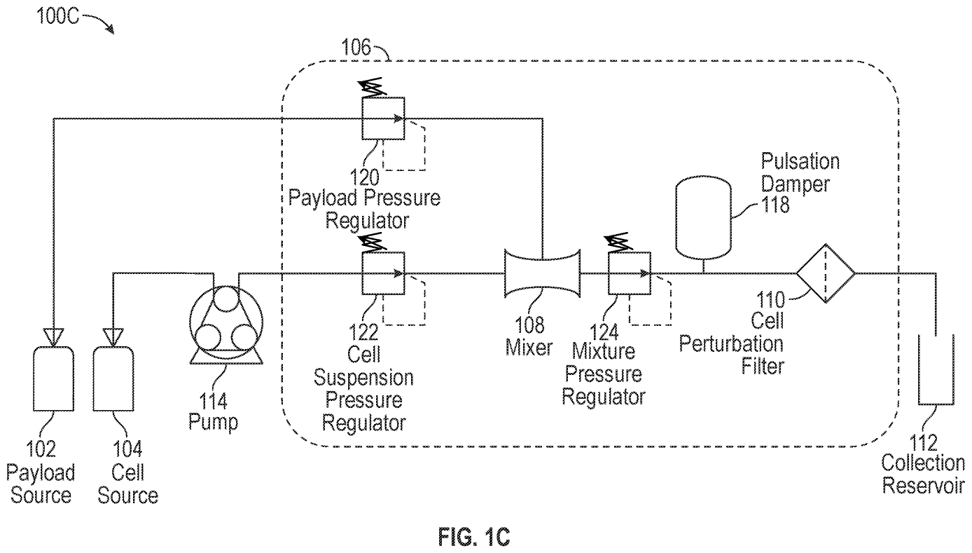

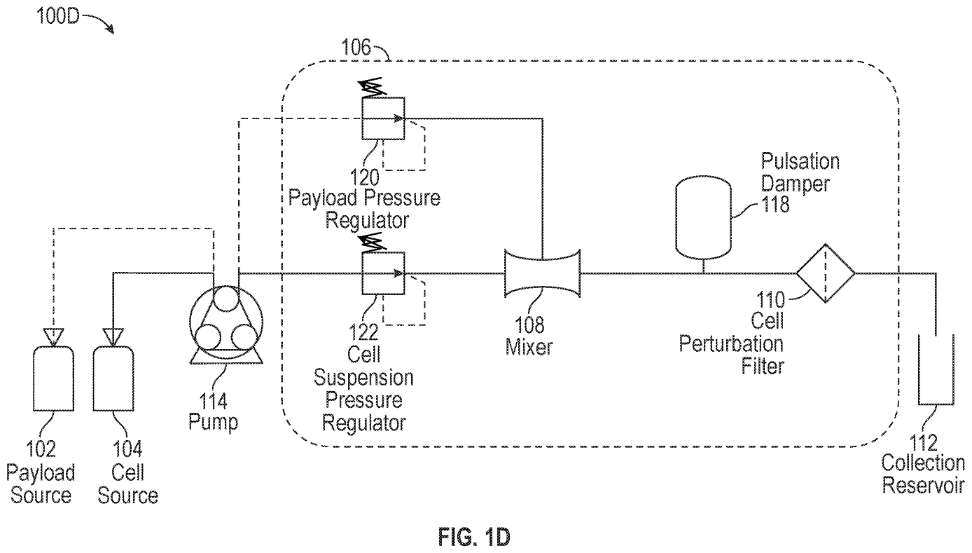

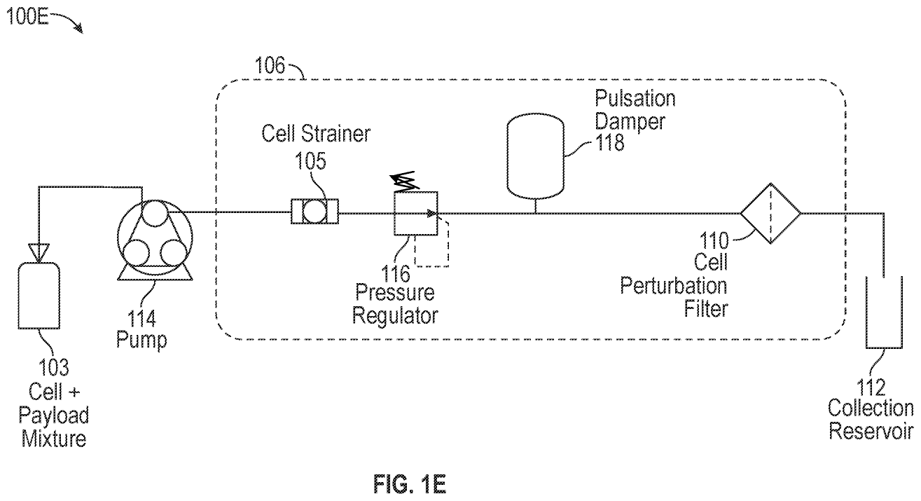

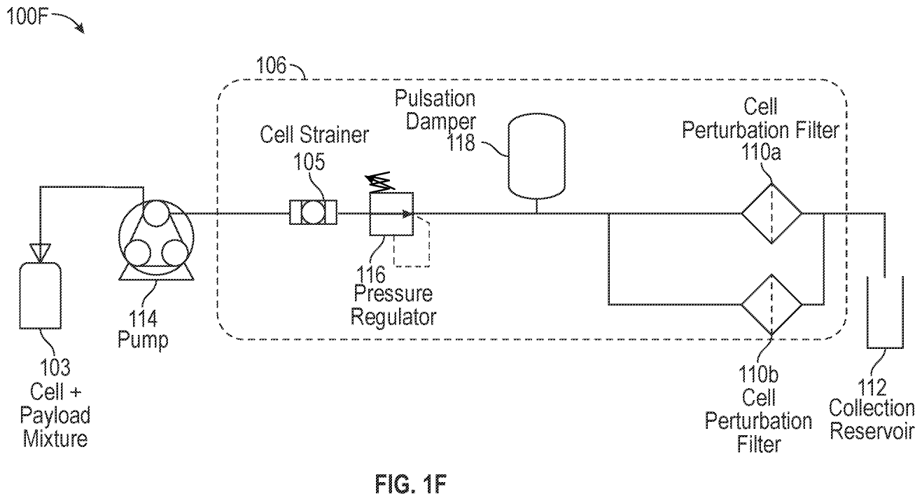

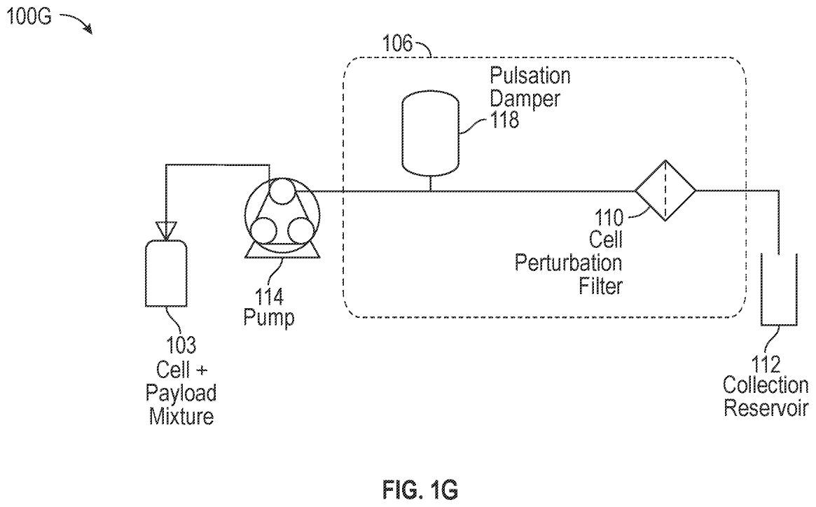

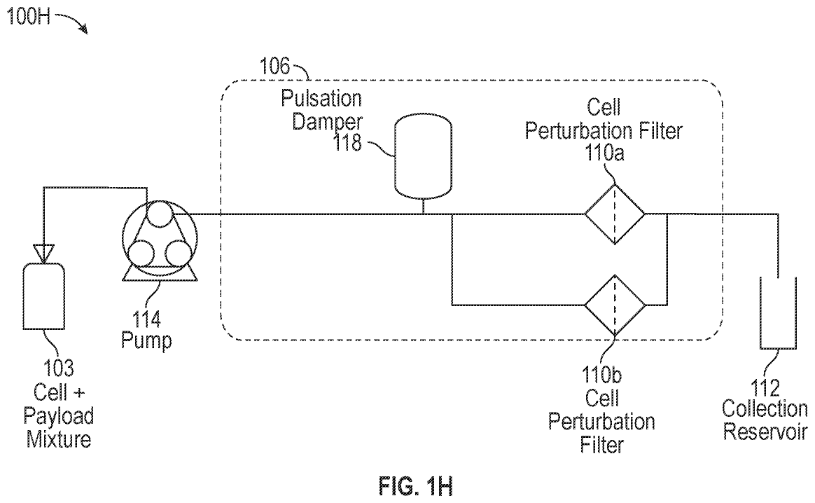

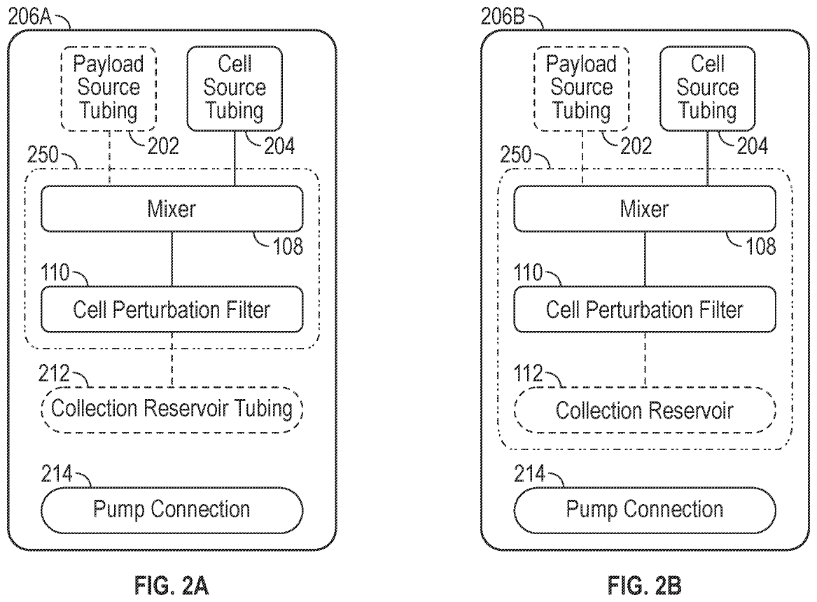

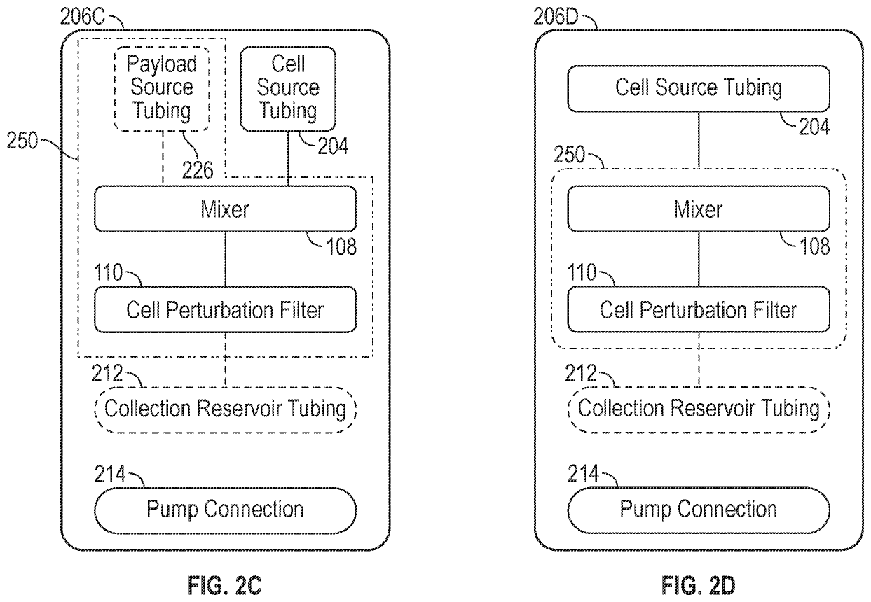

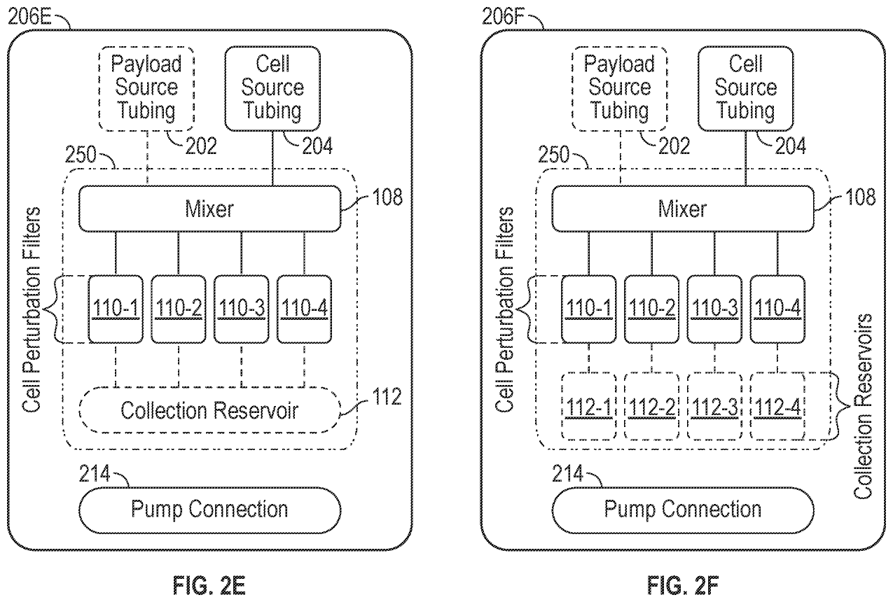

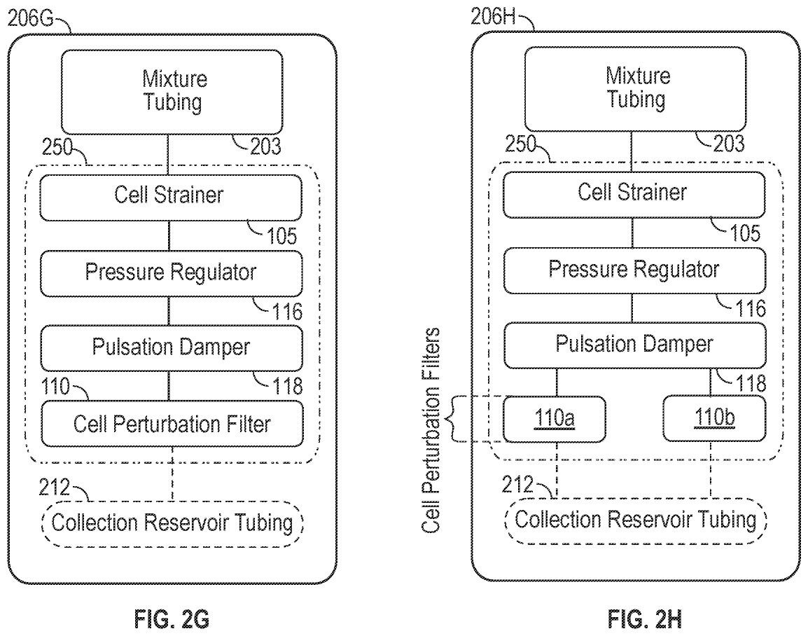

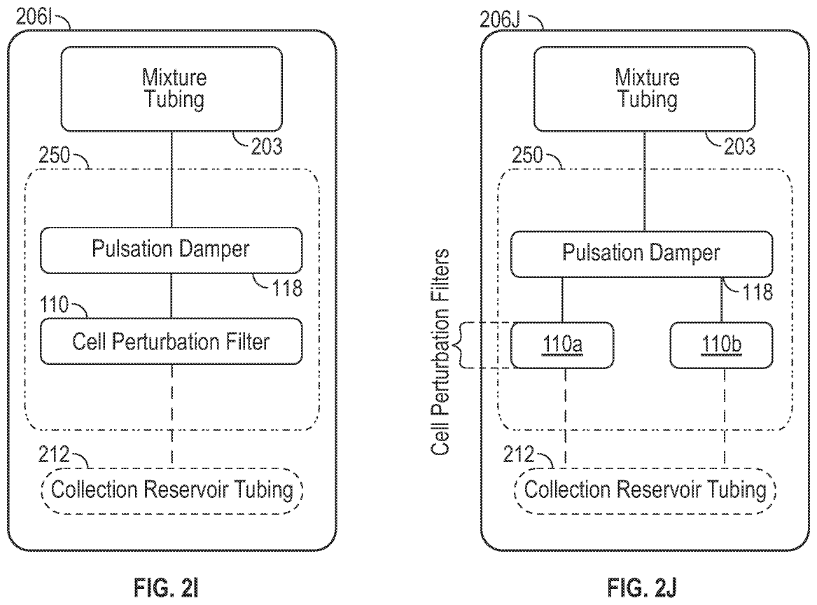

The description of FIGS. 1A-1H provided below primarily describes example systems for mechanoporation of cells, which can be used in conjunction with the methods and devices described herein. As mentioned above, the mechanoporation systems described herein may comprise a tubing set, examples arrangements of which are described below in relation to FIGS. 2A-2J.

FIGS. 1A-1H illustrate various mechanoporation systems for delivering a payload into cells. For example, each of the mechanoporation systems 100A, 100B, 100C, and 100D (collectively referred to hereinafter as mechanoporation system 100) illustrated in FIGS. 1A, 1B, 1C, and 1D, respectively may comprise at least a payload source 102, a cell source 104, a tubing set 106 comprising a mixer 108 and one or more cell perturbation filters 110, one or more collection reservoirs 112, and one or more pumps 114. In some embodiments, mechanoporation system 100 may additionally comprise one or more pressure regulators (e.g., pressure regulator 116, payload pressure regulator 120, cell suspension pressure regulator 122, and/or mixture pressure regulator 124) and/or a pulsation damper 118. In some embodiments, mechanoporation system 100 may not comprise a pressure regulator, as illustrated in FIGS. 1G-1H. In some embodiments, mechanoporation system 100 may not comprise a pulsation damper. In some embodiments, mechanoporation system 100 may not comprise a mixer 108, as illustrated in FIGS. 1E-1H. For example, as shown in FIGS. 1E-1H, the cell suspension and payload may be provided to system 100 in a mixed state (i.e., cell and payload mixture 103).

Payload source 102 may comprise a solution comprising a payload (e.g., hereinafter referred to as a “payload”). The payload for use in mechanoporation system 100 as well as the concentration of the payload may be dependent on the type of cells in the cell suspension and the intended cell therapy. The payload may include but is not limited to one or more of DNA, RNA (e.g., mRNA, siRNA, saRNA, RNA, miRNA, etc.), proteins, small molecules, peptides, nanoparticles, viruses, synthetic materials, a cell lysate comprising an antigen, and/or complexes (e.g., RNP, protein CAS plus guide RNA, etc.). The aforementioned payloads may function biologically as transcription factors, chemokines, cytokines, surface receptors, other intracellular or extracellular proteins, inhibitors, or enhancers of any of the above, survival factors, cryoprotectants, prime editors, antibodies, enzymes, and/or any combinations of the above.

The concentration of the payload in a cell suspension may vary based on the type of payload and may be less than or equal to 0.01 mg/mL, 0.1 mg/mL, 0.5 mg/mL, 1 mg/mL, 1.25 mg/mL, 1.5 mg/mL, 1.75 mg/mL, or 2 mg/mL. In some embodiments, the concentration of the payload in a cell suspension may be greater than or equal to 0.01 mg/mL, 0.1 mg/mL, 0.5 mg/mL, 1 mg/mL, 1.25 mg/mL, 1.5 mg/mL, 1.75 mg/mL, or 2 mg/mL.

Payload source 102 may comprise a flexible plastic bag, vial, bottle, vessel, ampule, jar, or other container suitable for containing the payload. Although illustrated external to tubing set 106 in each of FIGS. 1A-1D, it is contemplated that payload source 102 may be provided within (e.g., as a component of) tubing set 106. For example, rather than being an external source fluidly connected to tubing set 106, the payload may be contained in a payload reservoir (e.g., payload reservoir 226 illustrated in FIG. 2C) fluidly connected to mixer 108 within tubing set 106. In another example, the payload may be contained in mixer 108 of tubing set 106. In another example, the payload may be contained in a collection reservoir 112 disposed within tubing set 106 (e.g., illustrated at least in FIG. 2B). That is, mechanoporation system 100 may not comprise an external payload source 102. In each of these aforementioned embodiments, it is contemplated that the payload (e.g., disposed in an external payload source 102, in mixer 108, in payload reservoir 226, and/or in collection reservoir 112) may be combined with the cell suspension before, during, or after the cell suspension is passed through the one or more cell perturbation filters 110.

Cell source 104 may comprise a cell suspension. The cell suspension may comprise cells and a buffer solution. For example, the cells may comprise a mixture of cells in a physiological saline solution or physiological medium other than blood. The cells in the cell suspension may include but are not limited to a mixed cell population (e.g., whole blood) or a purified cell population (e.g., a purified nucleate or anucleate cell population). The cells in the cell suspension may include but are not limited to anucleate cells, (e.g., red blood cells such as erythrocytes or reticulocytes, platelets, etc.), nucleate cells (e.g., immune cells, PBMCs, T cells, NK cells, iPSCs, HSCs, etc.), liposomes, exosomes, etc. The cell suspension may comprise an aqueous solution including a cell culture medium (e.g., DMEM, RMPI, OptiMEM, etc.), phosphate-buffered saline (PBS), salts, sugars, growth factors, surfactants, lubricants, amino acids, proteins, cell cycle inhibitors, vitamins, etc.

The cell suspension may comprise a cell concentration between about 1.0×10¹ cells/mL and 1.0×10¹² cells/mL. Cell source 104 may comprise a flexible plastic bag, vial, bottle, vessel, ampule, jar, or other container suitable for containing the cell suspension. In another example, cell source 104 may not comprise a separate vessel containing the cell suspension, but rather tubing set 106 may be configured to receive the cell suspension from an upstream, fluidly connected cell therapy production system, cell washing system, filtration system, etc. For example, one or more of the aforementioned systems may process the cell suspension prior to providing the suspension to tubing set 106 by surface marker-based separation (e.g., Miltenyi), centrifugation/flow-based separation (e.g., elutriation), buffer exchanges, cell washing, activations, expansions, or other biological variations. As noted above and illustrated in FIGS. 1E-1H, in some examples, the tubing set 106 may receive a cell and payload mixture 103 from an upstream cell processing system that has mixed the cell suspension and the payload.

One or more of the payload and/or cell suspension may comprise a solution intended to prime tubing set 106. For example, the payload may comprise a cell buffer and/or payload buffer. In some embodiments, mechanoporation system 100 may be used without priming (or wetting). For example, tubing set 106 may be pre-wetted (e.g., the tubing set may be provided with wetting fluid disposed within the set).

As illustrated in mechanoporation system 100A of FIG. 1A, payload source 102 may be fluidly connected to cell source 104 to receive the cell suspension from cell source 104. For example, one or more pumps 114 may be configured to move the cell suspension from cell source 104 and into payload source 102. The one or more pumps 114 may instead or additionally be used to pressurize mixer 108 (e.g., by providing air pressure), which upon reaching a threshold pressure inside the mixer 108 can push the mixture through the filter 110. The one or more pumps 114 may be configured to provide positive pressure or negative pressure (e.g., a vacuum) to move the cell suspension, payload, and/or mixture through system 100. For example, negative pressure from one or more pumps 114 may “pull” one or more of the aforementioned solutions through the system, whereas positive pressure may “push” or drive the solutions through the system. The one or more pumps 114 may comprise a single pump 114. Pumps 114 may include but are not limited to peristaltic pumps, syringe pumps, diaphragm pumps, positive displacement pumps, gear lobes, etc. More generally, the term “pump” may further be used herein to refer to manual syringes, pipettes, pistons, gas cylinders, and/or other fluid motivators (e.g., gravity). In some embodiments, the one or more pumps 114 may be integrated within existing filtration systems, isolation systems, elutriation systems, cell washing systems, and/or end-to-end cell therapy production systems. For example, filtration, isolation, and/or elutriation systems can be used to separate cells prior to further cell processing. The systems may comprise functionalities to control and/or monitor one or more parameters of the system and/or solution, such as pressure, temperature, flow rate, cell viability, etc. The control and/or monitoring of the aforementioned parameters may be extended for use in mechanoporation system 100, at least because in attaching tubing set 106 to existing pumps 114, a closed system can be maintained.

Example filtration and/or elutriation systems include but are not limited to tangential flow filtration (TFF) systems, (e.g., Repligen KrosFlo® KR2i TFF system, Sartorius Ambr® Crossflow TFF system, Sartorius Sartoflow® Smart TFF system, etc.), standard filtration systems (e.g., Fresenius Cue® Cell Processing System), spinning membrane filtration, (e.g., Fresenius Kabi Lovo® Automated Cell Processing System), and elutriation systems (e.g., Gibco™ Cell Therapy Systems (CTS™) Rotea Counterflow Centrifugation System, Terumo Elutra® Cell Separation System, etc.). Example cell isolation systems include but are not limited to the Miltenyi CliniMACS Prodigy® instrument and StemCell cell isolation systems (e.g., immunomagnetic cell separation systems such as EasySep™, RoboSep™ and StemSep™, immunodensity cell separation systems such as RosetteSep™ and SepMate™, etc.). Example end-to-end cell therapy production systems include but are not limited to the Lonza Cocoon® Platform. Thus, as described below, tubing set 106 may be configured such that it can be connected to a variety of pumps, filtration systems, isolation systems, cell therapy production systems, and/or elutriation systems. Stated otherwise, tubing set 106 may be universally attachable to various different pumps, filtration systems, isolation systems, cell therapy production systems, and/or elutriation systems. In some embodiments, tubing set 106 may be integrated into (e.g., as a component of) one or more of the aforementioned pumps, filtration systems, isolation systems, cell therapy production systems, and/or elutriation systems.

In some embodiments, tubing set 106 may comprise one or more interchangeable components (e.g., tubing, connectors, dampers, pressure regulators, mixers, collection reservoirs, filters, etc.) that can be selected for use in system 100 based on the functionalities, volume requirements, pressure requirements, and/or connection types of the one or more pumps 114. One or more of the aforementioned components may instead or additionally be selected for use in system 100 based on requirements of the payload and/or the cell suspension, such as based on characteristics of the cells, the volume of the payload and/or cell suspension, etc.

The pumps 114 may be configured to provide a pressure (e.g., a pulsed pressure) to the mechanoporation system 100 between at least 2 psi and 25 psi. For example, the pressure provided by one or more pumps 114 may be greater than or equal to at least about 1 psi, 5 psi, 10 psi, 15 psi, 20 psi, 25 psi, 30 psi, or 35 psi. In some embodiments, the pressure provided by one or more pumps 114 may be less than or equal to at least about 1 psi, 5 psi, 10 psi, 15 psi, 20 psi, 25 psi, 30 psi, or 35 psi. System 100 may be configured to control or modify (e.g., step-down) the pressure provided by the one or more pumps 114 (e.g., using one or more valves, etc.) such that the pressure gradient across the one or more cell perturbation filters 112 is within the intended range (e.g., between 2 psi and 15 psi, such as 5 psi, 7.5 psi, 10 psi, 12.5 psi, etc.). In some examples, the pressure gradient across the one or more filters 110 may be less than 2 psi, such as 1.5 psi, 1 psi, 0.5 psi, or 0.25 psi. In some embodiments, the pressure gradient across the one or more filters 110 may be greater than 15 psi, such as 17.5 psi, 20 psi, 22.5 psi, 25 psi, 30 psi, or more.

The one or more pumps 114 may be configured to pump (e.g., move) a total volume between at least 1 mL and 500 mL into tubing set 106. For example, one or more pumps 114 may drive at least 1 mL, 50 mL, 100 mL, 200 mL, 300 mL, 400 mL, or 500 mL of fluid into tubing set 106. In some embodiments, the one or more pumps 114 may be configured to pump a minimum volume of 100 μL, 250 μL, 500 μL, or 750 μL. In some embodiments, the one or more pumps 114 may be configured to pump a total volume between at most 500 mL and 5 L. For example, one or more pumps 114 may drive at most 500 mL, 1 L, 2 L, 3 L, 4 L, or 5 L of fluid into tubing set 106. In some embodiments, the system 100 as described herein may be adapted for allogeneic production and/or bioprocessing, and thus the one or more pumps 114 may be configured to pump a volume greater than 5 L, such as 10 L, 100 L, or 1000 L.

The one or more pumps 114 may pump the cell mixture (e.g., the cell suspension and/or the payload) through the tubing set 106 at a flow rate between 5 mL/min and 100 mL/min. For example, the one or more pumps 114 may pump the cell mixture through the tubing set at a flow rate greater than or equal to about 5 mL/min, 10 mL/min, 15 mL/min, 20 mL/min, 25 mL/min, 30 mL/min, 40 mL/min, or 50 mL/min. In some examples, the one or more pumps 114 may pump the cell mixture through the tubing set at a flow rate less than or equal to about 25 mL/min, 30 mL/min, 40 mL/min, 50 mL/min, 60 mL/min, 70 mL/min, 75 mL/min, 80 mL/min, 90 mL/min, or 100 mL/min.

The one or more pumps 114 may be connected to a tubing set 106 comprising at least a mixer 108 and one or more cell perturbation filters 110. Thus, the one or more pumps 114 may be configured to move the payload and/or the cell suspension from the payload source 102 and/or the cell source 104 into the mixer 108. For example, with reference to FIGS. 2A-2F, tubing set 206A-F (understood to be equivalent to tubing set 106 in FIGS. 1A-1D) may comprise a pump connection portion 214 configured to connect to one or more pumps 114. In some embodiments, pump connection portion 214 may be substantially the same portion of tubing set 206 as the cell source tubing portion 204, at least because the one or more pumps 114 move the cell suspension into modules of tubing set 206. For example, in tubing sets 206G-J illustrated in FIGS. 2G-2J, the mixture tubing portion 203 is understood to encompass the connection between the one or more pumps (e.g., a cell processing system comprising the one or more pumps) and the tubing set 206.

As shown in mechanoporation system 100A illustrated in FIG. 1A, one or more pumps 114 may be configured to move a single stream comprising the payload and the cell suspension into mixer 108. In another example, with reference to mechanoporation systems 100B and 100C illustrated in FIGS. 1B and 1C, respectively, one or more pumps 114 may pump only the cell suspension from cell source 104 into mixer 108. In these embodiments, the one or more pumps 114 may not be connected to a portion of tubing set 106 fluidly connected to payload source 102. Rather, the flow of the payload from payload source 102 may be actuated using, for example, a pressure-dependent actuation device. In another example, mechanoporation system 100 may not comprise an external payload source 102 (e.g., at least because as described above the payload may be contained within tubing set 106), thus the one or more pumps 114 may only be connected to and configured to move the cell suspension from cell source 104 into tubing set 106.

In yet another example, with reference to mechanoporation system 100D illustrated in FIG. 1D, one or more pumps 114 may be configured to pump the payload from payload source 102 and the cell suspension from cell source 104 in separate yet parallel tubing portions. Each of payload source 102 and cell source 104 may be individually fluidly connected to mixer 108 (e.g., via individual tubing portions). In some embodiments, the parallel tubing portions may be fused at least to facilitate passing the tubing at substantially the same time through the one or more pumps 114.

In yet another example, with reference to mechanoporation systems 100E, 100F, 100G, and 100H illustrated in FIGS. 1E-1H, respectively, the one or more pumps may be configured to pump a cell and payload mixture 103 into the one or more cell perturbation filters 110. As will be described in greater detail below, in these examples, at least a portion of the mixture 103 may travel in and out of the pulsation damper 118 prior to reaching the cell perturbation filter(s) 110. As illustrated in FIGS. 1E-1F, the tubing set 106 may include a pressure regulator 116 that prevents the mixture 103 from flowing into the cell perturbation filter 110 until conditions appropriate for cell mechanoporation are reached in the tubing set 106.

As noted above, mechanoporation system 100 may comprise one or more pressure regulators 116. For example (with reference to the example mechanoporation system 100A illustrated in FIG. 1A), in the instance cell source 104 is fluidly connected to payload source 102 to provide the cell suspension to payload source 102, the system may comprise a single pressure regulator 116 fluidly connected to payload source 102. Systems in which a cell and payload mixture 103 is provided to the tubing set 106 (e.g., described with respect to FIGS. 1E-1F) may also include a single pressure regulator 116. Pressure regulator 116 may be provided as a component of tubing set 106. Pressure regulator 116 may be provided as separate from tubing set 106 such that it may be configured to fluidly connect to tubing set 106. Pressure regulator 116 may include but is not limited to active flow-controlling valves, passive flow-controlling valves, check valves, flow-limiting nozzles, back-pressure regulators, pressure-regulating limit valves, springs, tubing portions comprising a tortuous path (e.g., tubing comprising flow-limiting geometries), etc. In some embodiments, pressure regulator 116 may be non-invasive such that the component may not be fluidly connected to payload source 102 and rather may act externally to the system (e.g., to tubing set 106) to control pressure in the system. As mentioned above, in some embodiments, mechanoporation system 100 may not include any pressure regulators. For example, FIGS. 1G-1H depict mechanoporation systems 100G, 100H that may not include a pressure regulator within the tubing set 106. In some embodiments, the pressure may be regulated via operating parameters of an upstream cell processing system in addition to or instead of necessitating a pressure regulator component within the tubing set 106. In some examples, the pressure within the mechanoporation system 100 may be regulated indirectly by controlling the flow rate at the upstream cell processing system.

Pressure regulators 116 may comprise one or more sensors, such as pressure sensors, flow sensors, temperature sensors, etc. One or more of the sensors may require a power source, such as an external power source (e.g., a power supply) and/or an internal power source (e.g., a battery). Example batteries may include but are not limited to button-cells (e.g., CR2032) or other standard cylindrical alkaline batteries (e.g., AA, AAA, etc.). In the instance tubing set 106 requires a power source to operate one or more components of the system, an internal power source (e.g., a single-use battery) may be used, at least because it can allow tubing set 106 to be disposable. In some embodiments, the power supply may be a semi-reusable and/or fully reusable power supply, such as a rechargeable battery module (e.g., lithium-polymer or LiPo battery) that may be configured to removably connect to system 100 (e.g., to tubing set 106). The reusable power supply may therefore be reused for multiple disposable tubing sets described herein. In some embodiments, tubing set 106 may not include sensors, and as discussed herein may utilize one or more sensors of the pumping system (e.g., a filtration and/or elutriation system comprising one or more pumps 114) to monitor and/or control parameters of system 100. In some embodiments, tubing set 106 may not comprise a power source (e.g., an external and/or internal power source).

Mechanoporation system 100 may comprise one or more devices configured to control and/or display data from the one or more pressure regulators 116. For example, the devices may include one or more monitors, laptops, tablets, cellular phones, or other devices configured to receive and display data and/or optionally control parameters of the pressure regulators 116. The one or more devices may communicate with the pressure regulators 116 via a wired and/or wireless (e.g., Wi-Fi, Bluetooth, etc.) connection.

FIGS. 1B-1D illustrate additional contemplated arrangements of payload source 102, cell source 104, and corresponding pressure regulators of each. For example, as described in greater detail below, each of payload source 102 and cell source 104 may be fluidly connected to mixer 108 of tubing set 106. In this instance, mechanoporation system 100 may comprise a plurality of pressure regulators, including a payload pressure regulator 120 and/or a cell suspension pressure regulator 122. Payload pressure regulator 120 may be fluidly connected to payload source 102. Cell suspension pressure regulator 122 may be fluidly connected to cell source 104. Payload pressure regulator 120 and/or cell suspension pressure regulator 122 may comprise any one or more features of pressure regulator 116 described above with respect to FIG. 1A. For example, one or more of payload pressure regulator 120 and/or cell suspension pressure regulator 122 may be provided as a component of and/or separate from tubing set 106.

In some embodiments, mechanoporation system 100 may additionally or instead include one or more mixture pressure regulators 124. Mixture pressure regulator 124 may be fluidly connected to mixer 108 to receive a mixture from the mixer 108. Mixture pressure regulator 124 may comprise any one or more features of pressure regulator 116, payload pressure regulator 120, and/or cell suspension pressure regulator 122 as described above.

As mentioned above, mechanoporation system 100 may comprise a tubing set 106, the tubing set 106 comprising at least a mixer 108 and one or more cell perturbation filters 110. In some embodiments, tubing set 106 may comprise a pulsation damper 118. In some embodiments, the pulsation damper 118 and the mixer 108 may be encompassed in a single component in the tubing set 106. Mixer 108 may be configured to mix the payload and the cell suspension to produce a mixture. The one or more cell perturbation filters 110 may be configured to perturb membranes of cells of the mixture as the mixture passes through the one or more cell perturbation filters 110. Pulsation damper 118 may be configured to normalize pulsations from the one or more pumps 114. Each of the tubing set 106, mixer 108, cell perturbation filters 110, and pulsation damper 118 are described in greater detail below.

FIGS. 2A-2J illustrate block diagrams comprising various arrangements of at least a portion of the components that may be included in tubing set 106. Tubing set 106 is understood to share any one or more features with tubing sets 206A, 206B, 206C, 206D, 206E, 206F, 206G, 206H, 206I, and 206J (hereinafter collectively referred to as tubing set 206). The block diagram of tubing sets 206 illustrated in FIGS. 2A-2F is not intended to be limiting; for example, as stated above, the tubing set may comprise one or more pressure regulator components and/or a pulsation damper component. For example, FIGS. 2G-2H depict tubing sets 206G, 206H that include a pressure regulator 116 and pulsation damper 118. In another example, FIGS. 2I-2J depict tubing sets 206I, 206J that includes only a pulsation damper 118 in addition to the one or more cell perturbation filters 110. Moreover, it is to be understood that any suitable combination of features from the various arrangements of components in tubing set 206 can be contemplated. For example, although not explicitly illustrated, a tubing set 206 may be provided that comprises a payload reservoir 226 and a collection reservoir 112 within a housing 250 of the tubing set.

As shown at least in FIG. 2A, tubing set 206 may comprise a payload source tubing portion 202 configured to fluidly connect to payload source 102 comprising the payload. Payload source tubing portion 202 may fluidly connect to mixer 108. For example, one or more pumps 114 may be configured to move the payload from payload source 102, through payload source tubing portion 202, and into mixer 108. As mentioned above, the one or more pumps 114 may be configured to connect to a pump connection portion 214 of tubing set 206. In some embodiments, payload source tubing portion 202 may comprise a payload pressure regulator (e.g., payload pressure regulator 120).

Tubing set 206 may comprise a cell source tubing portion 204 configured to fluidly connect to cell source 104. Cell source tubing portion 204 may fluidly connect to mixer 108. One or more pumps 114 be configured to move the cell suspension from cell source 104, through cell source tubing portion 204, and into mixer 108. Cell source tubing portion 204 may comprise a cell suspension pressure regulator (e.g., cell suspension pressure regulator 122). Cell source tubing portion 204 may comprise one or more leukocyte reduction filters and/or cell strainers, described in greater detail below.

As shown in FIGS. 2G-2J, tubing set 206 may comprise a mixture tubing portion 203 configured to fluidly connect to an upstream cell processing system (or other pump) that has mixed a cell suspension and payload. Mixture tubing portion 203 may fluidly connect the one or more cell perturbation filters 110. In some examples, the tubing set 206 (e.g., the housing 250 of the tubing set 206) may include one or more of a pressure regulator 116 and/or pulsation damper 118 disposed along the mixture tubing portion 203 and upstream of the one or more cell perturbation filters 110. The tubing sets 206 illustrated in FIGS. 2G-2H may not include a pump connection portion 214, at least because the pump connection between the tubing set 206 and an upstream cell processing system (or other pump) may be encompassed by the mixture tubing portion 203.

Tubing set 206 (e.g., payload source tubing portion 202, mixture tubing portion 203, and/or cell source tubing portion 204) may comprise one or more portions of tubing. The portions of tubing may comprise one or more of polyvinyl chloride (PVC) tubing, C-flex tubing, and/or other tubing manufactured by MasterFlex and/or Watson Marlow. Tubing set 206 may be configured to fluidly connect to one or more of cell source 104 and/or payload source 102 (e.g., including an upstream cell processing system or other pump) using tube weld connections. Tubing set 206 may instead or additionally be configured to fluidly connect to one or more of cell source 104 and/or payload source 102 (e.g., including an upstream cell processing system or other pump) using connectors. For example, cell source tubing portion 204 may fluidly connect to cell source 104 using a tube welded connection and/or one or more connectors. Likewise, payload source tubing portion 202 may fluidly connect to payload source 102 using a tube welded connection, swaged connection, and/or one or more connectors. In another example, the mixture tubing portion 203 may fluidly connect to an upstream cell processing system using a tube welded connection, swaged connection, and/or one or more connectors. The connectors may comprise one or more Luer connectors, Y connectors, quick disconnect connectors, barbed connectors, straight connectors, suction connectors, threaded connectors, push-to-connect connectors, and/or T connectors.

One or more tubing portions of tubing set 206 may comprise a diameter less than or equal to 2 mm, 3 mm, 3.25 mm, 3.5 mm, 3.75 mm, 4 mm, or 5 mm. The diameter of one or more tubing portions may be greater than or equal to 2 mm, 3 mm, 3.25 mm, 3.5 mm, 3.75 mm, 4 mm, or 5 mm. The diameter may be configured to correspond with that of one or more aforementioned connectors, such as a standard Luer connector (e.g., about 3.7 mm).

Tubing set 206 may comprise a housing 250 configured to contain at least the mixer 108 and one or more cell perturbation filters 110 in a single rigid body. In some embodiments, housing 250 may contain additional or alternative components including but not limited to pulsation dampers 118, pressure regulators (e.g., pressure regulator 116, payload pressure regulator 120, cell suspension pressure regulator 122, and/or mixture pressure regulator 124), and fluid reservoirs. For example, as shown at least in tubing sets 206B and 206F in FIGS. 2B and 2F, respectively, housing 250 may comprise one or more collection reservoirs 112 configured to collect the perturbed cell mixture. In another example illustrated in tubing set 206C of FIG. 2C, housing 250 may comprise a payload reservoir 226 configured to contain the payload. As described above, in some embodiments the mechanoporation system 100 may not comprise an external payload source 102, and rather the payload may be contained in tubing set 106. Accordingly, FIG. 2C illustrates an example tubing set 206C in which a payload reservoir 226 can be contained within housing 250 of tubing set 206. In another example illustrated in FIGS. 2G-2J, the housing 250 does not comprise a mixer 108 per se, but rather includes a pulsation damper 118. In some examples, the pulsation damper 118 may mix the mixture as it passes through the housing 250.

Housing 250 may be removably connected to tubing set 206. For example, housing 250 may comprise a single rigid body configured to attach to one or more tubing portions of tubing set 206, thereby fluidly connecting external components of mechanoporation system 100 (e.g., payload source 102, cell source 104, pump 114, and/or collection reservoir 112) to components contained within housing 250. Housing 250 may define part of the flow path of mechanoporation system 100 for delivering a payload to a cell. For example, the cell suspension and payload may flow from payload source 102 and/or cell source 104 of system 100 and into one or more inlets of housing 250. The cell suspension and payload may flow sequentially through mixer 108 and the one or more cell perturbation filters 110 within the housing before flowing out of an outlet of housing 250 and into a collection reservoir 112. In some examples, a cell and payload mixture may flow into an inlet of the housing 250, through the one or more cell perturbation filters 110, and out of an outlet of housing 250 to a collection reservoir 112 fluidly connected to the outlet of the housing 250.

Housing 250 and more generally tubing set 206 (and therefore tubing set 106) may be a single-use, disposable device. In some embodiments, housing 250 may comprise one or more components made of metal, plastic, polymers, and/or glass. For example, housing 250 may comprise one or more components made of polycarbonate, polypropylene, and/or polymethyl methacrylate. Tubing set 206 and therefore housing 250 may be sterilizable, for example, using heat (steam or dry heat), radiation (e.g., gamma), ethylene oxide gas, vaporized hydrogen peroxide, and/or other sterilization methods (e.g., chlorine dioxide gas, vaporized peracetic acid, and nitrogen dioxide).

Mixer 108 may be fluidly connected to payload source 102 and cell source 104. For example, as shown in mechanoporation system 100A of FIG. 1A, cell source 104 may be fluidly connected to payload source 102 to provide the cell suspension to payload source 102, and payload source 102 may be fluidly connected to mixer 108 to provide the cell suspension and the payload to the mixer. In another example, as shown in mechanoporation systems 100B, 100C, and 100D illustrated with respect to FIGS. 1B-1D, respectively, each of payload source 102 and cell source 104 may be individually fluidly connected to mixer 108. In some examples, the mixer 108, or more generally, the functionality of mixing the payload and the cell suspension, may be encompassed within the upstream cell processing system (e.g., pump or filtration system). For example, mechanoporation systems 100E-100H described herein with respect to FIGS. 1E-1H depict a cell and payload mixture 103 provided to the tubing set 106 by an upstream cell processing system (including the one or more pumps 114).

As discussed herein, tubing set 106 may comprise the payload rather than system 100 comprising an external payload source 102. For example, as shown in FIG. 2C, housing 250 of tubing set 206C may comprise a payload reservoir 226 fluidly connected to mixer 108. In some embodiments, housing 250 may comprise a seal, valve, or other fluid-regulating mechanism between payload reservoir 226 and mixer 108 to contain the payload in the reservoir until mixing can occur. As discussed herein, payload reservoir 226 may be fluidly connected in the system to combine the payload and the cell suspension before, during, or after passing the mixture through the one or more cell perturbation filters 110. Stated otherwise, although not explicitly illustrated, the payload may be combined with the cell suspension subsequently to passing the cell suspension through the one or more cell perturbation filters 110.

In some embodiments, mixer 108 may be configured to contain the payload. FIG. 2D illustrates an example tubing set 206D which may not comprise an external payload source 102 and/or a separate payload reservoir 226. Rather, the payload may be contained within mixer 108. Each of the tubing sets 206 comprising payload reservoir 226 and/or mixer 108 configured to contain the payload may contain the payload within housing 250, as mentioned above. As discussed herein, mixer 108 optionally containing the payload may be fluidly connected within tubing set 206 subsequent to the one or more cell perturbation filters 110 such that the cell suspension and payload can be mixed after perturbing the cells in the cell suspension. In some embodiments, tubing set 206 may be provided with the payload stored within the tubing set, such as in payload reservoir 226 or mixer 108. In this instance, at least housing 250 of tubing set 206 may be stored before use in a temperature-controlled environment (e.g., a refrigerator) to maintain the efficacy of the payload.

The concentration of the cell mixture comprising the cell suspension and the payload (e.g., produced by the mixer 108 and/or provided to the tubing set 106 by an upstream cell processing system that has performed mixing) may be between 1 million cells/mL and 100 million cells/mL. For example, the concentration of the cell mixture may be greater than or equal to about 1, 2, 5, 10, 15, 20, 25, 50, or 75 million cells/mL. In some examples, the concentration of the cell mixture may be less than or equal to 2, 5, 10, 15, 20, 25, 50, 75, or 100 million cells/mL.

Mixer 108 may comprise a low-shear mixing configuration such as in-line agitating geometry and/or an intersecting flow path. Mixer 108 may be static in that it can mix the payload and the cell suspension without an agitation device or impeller, thus limiting any risk of affecting viability of the cells by limiting shear. In some embodiments, however, it can be contemplated that mixer 108 comprises an active mixing component such as an agitation device or impeller. In some embodiments, the active mixing device may require a power source (e.g., an external or internal power source, described above).

Mixer 108 may comprise a tubing portion or other closed vessel comprising agitating geometry, such as a spiral configuration, one or more obstacles, nozzle geometries including but not limited to Venturi pumps, eductors, jet mixers, rotating or otherwise-actuated flow outlets (e.g., driven by the working fluid pressure or flow, such as an aeolipile) configured to induce mixing of the payload and the cell suspension. Mixer 108 may instead or additionally comprise an intersecting flow path configured to intersect and therefore mix the individual payload and cell suspension feeding into mixer 108. For example, a cell source tubing portion and a payload source tubing portion may each be fluidly connected to mixer 108 at an angle from one another (e.g., an angle between 1° and 180°) that induces mixing of the payload and the cell suspension in mixer 108.

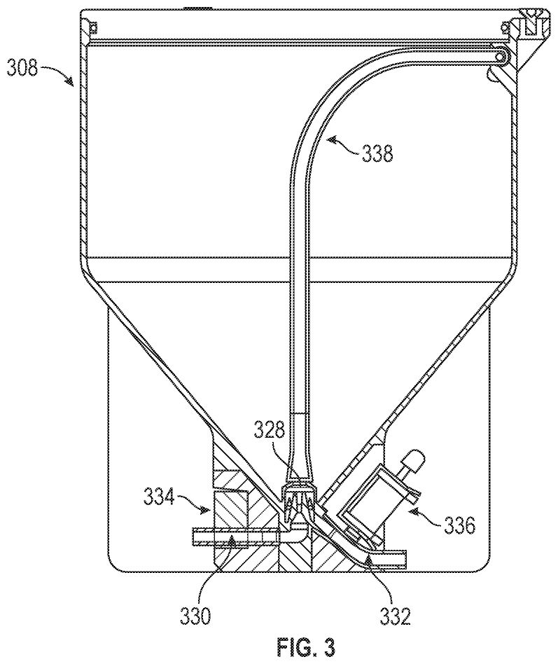

An example mixer 108 comprising an intersecting flow path includes Venturi mixers, an example of which is illustrated in FIG. 3. Venturi mixers are understood by one of ordinary skill in the art to comprise at least two inlet flow paths and a single outlet flow path. The Venturi mixer may comprise a flow-controlling valve dictated by pressure upstream from the mixer that can actuate the streams at the inlet flow paths. Thus, a Venturi mixer may be used to actuate the flow of the payload and the cell suspension from payload source 102 and cell source 104, respectively, and may also be used to controllably mix the payload and the cell suspension to produce the mixture. In each of the above-described embodiments, mixer 108 may be configured to produce a mixture comprising substantially a 1:1 ratio of cell suspension and payload. Moreover, mixer 108 described herein may be configured to account for air pressure present within system 100 to minimize damage to cells and/or dead volumes. For example, air pressure may pressurize mixer 108 to drive the fluid (e.g., the cell suspension and/or mixture comprising the cell suspension and payload) through mixer 108 and/or through one or more cell perturbation filters 110. Thus, mixer 108 may be configured to account for such air pressure that may be present within system 100 by selecting a specific orientation of agitating geometries in mixer 108 and/or valves, nozzles, etc. that provide the cell suspension and payload to mixer 108.

With reference to FIG. 3, a mixer may comprise a Venturi valve 328 disposed within a mixer vessel 308. In some embodiments, mixer vessel 308 may be provided with the payload contained within the vessel, such that Venturi valve 328 comprises a single external inlet 330 to provide the cell suspension to mixer vessel 308. Venturi valve 328 may comprise an internal inlet (not illustrated) to combine the payload contained within mixer vessel 308 with the cell suspension at the Venturi valve 328. Although not explicitly illustrated, as discussed herein, the mixer vessel 308 may instead be fluidly connected to more than one external fluid source (e.g., payload source 102 and cell source 104). For example, mixer vessel 308 may comprise an inlet disposed within an upper portion of the vessel to provide the payload to the mixer vessel 308, the inlet fluidly connected to an external payload source 102. The mixer vessel 308 may comprise a single outlet 332 to provide the mixture to a subsequent module (e.g., the one or more cell perturbation filters 110).

The Venturi mixer configuration illustrated in FIG. 3 may comprise one or more other components, such as a liquid level detector 334 and/or an outlet valve 336. Liquid level detector 334 may be configured to detect air (e.g., and therefore the absence of liquid) in inlet 330, wherein the presence of air may signify the completion of adding a cell suspension to the mixer vessel 308. Outlet valve 336 may be configured to allow the mixture to exit mixer vessel 308 based on a determination that the full volume of the cell suspension has been added to the mixer vessel 308 and mixed with the payload. For example, outlet valve 336 may be a pinch valve or other check valve configured to actuate after (e.g., instantaneously after, after a delay, etc.) the full volume of a cell suspension has been added to mixer vessel 308. In some embodiments, outlet valve 336 may be configured to actuate based on a pressure detected in the system (e.g., once a threshold pressure is achieved, the mixture may be released from mixer vessel 308). Outlet valve 336 may be configured to control the pressure and/or flow rate of the mixture as it is released from mixer vessel 308.

The Venturi valve 328 may be fluidly connected to a recirculating outlet 338 configured to guide a mixture comprising the cell suspension and the payload into the interior wall of mixer vessel 308. As shown in FIG. 3, recirculating outlet 338 may be configured to dispose the mixture at an upper portion of mixer vessel 308, for example, to minimize the presence of air pressure from inlet 330 creating bubbles or foaming within the system. Mixer vessel 308 may comprise a conical or shape, as shown, to facilitate the collection of the mixture within the tapered portion comprising mixture outlet 332. The scale of the various components (e.g., mixer vessel 308) illustrated in FIG. 3 is intended to be illustrative. As described herein, it is to be understood that mixer vessel 308 (and therefore mixer 108) may be of various volumetric capacities ranging from microliters to liters.

In some embodiments, tubing set 106 may comprise a pulsation damper 118 configured to normalize pulsations from one or more pumps 114. Pulsation dampers 118 can include flexible tubing and/or fluid reservoirs (e.g., a reservoir comprising a diaphragm) as described herein. For example, the pulsation damper 118 may be in the form of a static, one-end closed portion of tubing, e.g., arranged as a coiled (e.g., for preserving space) or other column of tubing. The pulsation damper 118 may be configured to adapt to fluctuations in air pressure that may be introduced to system 100 to maintain a constant overall system pressure. For example, the pulsation damper 118 may be configured to receive fluid (e.g., payload and cell suspension) and subsequently release the fluid downstream in the tubing set 106 to counteract pulsations introduced from an upstream pump 114 (e.g., a pump of a cell processing system). Pulsation damper 118 may be fluidly connected to mixer 108 to receive the mixture. Although illustrated subsequent to mixer 108 in FIGS. 1A-1D, it is to be understood that pulsation damper 118 may be disposed in mechanoporation system 100 in a different sequence from that which is illustrated. For example, pulsation damper 118 may be disposed in the system subsequent to one or more pumps 114 and/or subsequent to one or more cell perturbation filters 110.

In some embodiments, mechanoporation system 100 may not comprise a pulsation damper 118. For example, mixer 108 may be configured to normalize pulsations from the one or more pumps 114. Stated another way, in some examples, the mixer 108 may comprise features described herein with respect to the pulsation damper 118 such that the two components may be conflated to one component in the mechanoporation system 100. For example, the mixer 108 may include a one-end closed portion of tubing that can receive fluid (e.g., a cell suspension and payload) and subsequently release the fluid to counteract pulsations introduced from an upstream pump of a cell processing system. When the cell suspension and payload flow in and out of the tubing, they can be mixed to produce a mixture. In another example, one or more of payload source 102 and/or cell source 104 may be configured to normalize pulsations from the one or more pumps 114. In yet another example, in the instance tubing set 106 comprises a payload reservoir 226, the payload reservoir may be configured to normalize pulsations from the one or more pumps.

In some embodiments, tubing set 106 may comprise one or more leukocyte reduction filters and/or cell strainers 105, illustrated at least in FIGS. 1E-1F. For example, tubing set 106 may comprise one or more leukocyte reduction filters configured to remove white blood cells from a cell suspension in advance of the cell suspension (and payload) being passed through the one or more cell perturbation filters 110. Likewise, cell strainers may be configured to remove larger clumps (e.g., clumps within a size range of 40-70 μm) that may otherwise clog the one or more filters 110. The one or more leukocyte reduction filters and/or cell strainers 105 may be fluidly connected to cell source 104 to receive the cell suspension. In some examples, the one or more leukocyte reduction filters and/or cell strainers 105 may be fluidly connected to the cell and payload mixture 103 to remove unwanted aggregates from the mixture prior to passing the mixture through the cell perturbation filter(s) 110. The leukocyte reduction filter and/or cell strainer 105 may comprise pores sized such that the payload and desired components of the cell suspension can pass through. The one or more leukocyte reduction filters/cell strainers 105 may be provided separately from tubing set 106. For example, one or more leukocyte reduction filters and/or cell strainers 105 may instead or additionally be provided as a component of the one or more pumps 114 (e.g., more specifically, a known filtration or elutriation system, as described above).

One or more cell perturbation filters 110 may be fluidly connected to mixer 108 to receive the mixture. In the instance mechanoporation system 100 comprises a pulsation damper 118 (e.g., described above as optionally fluidly connected to mixer 108 to receive the mixture), the one or more cell perturbation filters 110 may be fluidly connected to the pulsation damper 118. As noted above, in some examples, the pulsation damper 118 may be embodied by the mixer 108 within the mechanoporation system 100, and the one or more cell perturbation filters 110 may receive the mixture from this mixer 108. The aforementioned one or more pumps 114 may be configured to move the mixture through the one or more cell perturbation filters 110. The one or more cell perturbation filters 110 may be configured to perturb membranes of cells of the mixture as the mixture passes through the filter. For example, the one or more cell perturbation filters 110 may comprise a porous surface, each pore of the porous surface configured to perturb membranes of cells as the cell suspension passes through the one or more cell perturbation filters 110. Example filters may include but are not limited to Cytiva Whatman™ Nuclepore™ Polycarbonate Hydrophilic Membrane sheets, Sterlitech Hydrophilic Polycarbonate Track Etch (PCTE) Membrane Filters, Aquamarijn low porosity microsieve Membranes™, or other commercially available filters comprising similar characteristics that are not otherwise explicitly provided herein but understood by one of ordinary skill in the art to be encompassed. In some examples, the one or more cell perturbation filters 110 may include specially constructed filter(s) for mechanoporation of cells within a specific range of sizes or to achieve particular performance metrics. For example, a size (e.g., diameter) and/or shape of the pores of the cell perturbation filter 110 may be selected to perturb cell membranes of a particular type or size of cell (or cells). The porosity (porous surface area), pore thickness (e.g., length), overall surface area of the cell perturbation filter 110, etc. may be selected to achieve efficacious cell mechanoporation.

As discussed herein, tubing set 106 comprising the one or more cell perturbation filters 110 may be configured to receive a volume between at least 10 mL and 50 mL. For example, the one or more cell perturbation filters 110 may be configured to receive a volume of at least 10 mL, 20 mL, 30 mL, 40 mL, or 50 mL. In some embodiments, the delivery portion may be configured to receive a volume between at most 250 mL and 500 mL. For example, the one or more cell perturbation filters 110 of the delivery portion may be configured to receive a volume of at most 250 mL, 300 mL, 350 mL, 400 mL, 450 mL, or 500 mL. As discussed herein at least with reference to the one or more pumps 114, system 100 (and therefore the one or more cell perturbation filters 110) may be configured to receive a volume less than 10 mL, such as 100 μL, 250 μL, 500 μL, 750 μL, 1 mL, 2.5 mL, 5 mL, or 7.5 mL. Likewise, in some embodiments, cell perturbation filters 110 may be configured to receive a volume greater than 500 mL, such as 750 mL, 1 L, 2.5 L, 5 L, 7.5 L, 10 L, or more.

The one or more cell perturbation filters 110 may comprise one or more materials such as synthetic or natural polymers, polycarbonate, silicon, silicon nitride, silicon oxide, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, and/or ceramic. The one or more cell perturbation filters 110 may comprise a coating, the coating comprising one or more materials such as Teflon, polyvinylpyrrolidone, an adhesive coating, surfactants, proteins, adhesion molecules, antibodies, anticoagulants, factors that modulate cellular function, nucleic acids, lipids, carbohydrates, and/or transmembrane proteins. In some embodiments, the one or more cell perturbation filters 110 may not comprise a coating.

The porous surface of the one or more cell perturbation filters 110 may comprise uniformly-sized pores or varying pore sizes throughout the surface. The pores of the one or more cell perturbation filters 110 may comprise one or more cross-sectional shapes including but not limited to circular, rectangular (e.g., square), elliptical, triangular, or another polygonal shape. In the instance the cross-section of the pores comprises a circular shape, the diameter of each of the one or more pores may be between 0.1 μm and 20 μm. For example, the diameter of a given pore may be about 0.1 μm, 0.5 μm, 1 μm, 2 μm, 5 μm, 10 μm, 15 μm, or 20 μm. The diameter of the each of the pores of the one or more cell perturbation filters 110 may be based on the diameter of the cells in the cell suspension. For example, the diameter of each of the pores may be a smaller diameter than a cell of a cell suspension passing through the filter, such that forcing the cell through the pore under pressure causes a perturbation in the membrane of the cell as the cell is constricted by the pore. The pore diameter may be between 10% and 99% of the diameter of the cells in a cell suspension, such as about 10%, 20%, 30%, 40%, 50%, 60%, 70%, 80%, 90%, 95%, or 99% of the diameter of the cells. It is to be understood that reference to a diameter of a cell in the cell suspension means the diameter of the cell in the cell suspension prior to being passed through the filter, e.g., as the cell approaches the filter, unless otherwise specified.

The porous surface of the one or more cell perturbation filters 110 may comprise a range of total pores. For example, the pores may cover between about 0.1% and about 60% of the total surface area of the filter. In some embodiments, the porous surface may comprise between about 1.0×10¹ to about 1.0×10¹⁰ pores per millimeter of surface area. The porous surface may in some embodiments comprise between about 1.0×10¹ to about 1.0×10¹⁵ pores per millimeter of surface area. The one or more cell perturbation filters 110 may have a cross-sectional shape comprising a circular, triangular, rectangular, elliptical, or other polygonal shape.

The one or more cell perturbation filters 110 may comprise a thickness, or length, defined by the axis by which the mixture travels along as it passes through the one or more cell perturbation filters 110. The thickness of the one or more filters 110 may be between about 0.1 μm and 1 mm. For example, the thickness of the one or more cell perturbation filters 110 may be less than or equal to about 0.1 μm, 0.5 μm, 1 μm, 5 μm, 10 μm, 50 μm, 100 μm, 500 μm, or 1 mm. In some embodiments, the thickness of the one or more cell perturbation filters 110 may be greater than or equal to about 0.1 μm, 0.5 μm, 1 μm, 5 μm, 10 μm, 50 μm, 100 μm, 500 μm, or 1 mm. Thus, given the thickness of the filter, the pores of the filter may comprise a 3-dimensional shape. In the instance a pore comprises a circular cross-section with a uniform diameter along the thickness of the filter, the 3-dimensional shape of the pore may comprise a cylinder. In the instance the diameter of the pore varies (e.g., increases or decreases) along the thickness of the filter, the 3-dimensional shape of the pore may be conical.

The one or more cell perturbation filters 110 may comprise a plurality of cell perturbation filters 110. The tubing set may comprise any number of cell perturbation filters 110, such as 1, 2, 3, 4, 5, 6, 7, 8, 9, 10 or more cell perturbation filters. For example, FIGS. 1F and 1H illustrates cell mechanoporation systems 100F and 100H in which the tubing set 106 includes a pair of cell mechanoporation filters 110a, 110b arranged in parallel, such that each filter 110a, 110b receives a portion of the cell and payload mixture 103 for mechanoporation. FIG. 2E illustrates a tubing set 206E comprising a plurality of cell perturbation filters 110 (e.g., filter 110-1, 110-2, 110-3, 110-4, collectively referred to herein as cell perturbation filters 110). The plurality of cell perturbation filters 110 may be disposed in parallel in tubing set 206E such that each filter of the plurality of cell perturbation filters 110 may be fluidly connected to mixer 108 (or in some embodiments pulsation damper 118) to receive a portion of the mixture, and to the one or more collection reservoirs 112 to provide the cell perturbed mixture to the reservoir. FIGS. 2H and 2J illustrate tubing sets 206H, 206J comprising a pair of cell perturbation filters 110a, 110b arranged in parallel such that each filter 110a, 110b is fluidly connected to an upstream cell processing system (via mixture tubing portion 203) to receive a portion of the cell and payload mixture. Although not explicitly illustrated in the Figures, it can be contemplated that one or more cell perturbation filters 110 may be fluidly connected in series.

Cell mechanoporation requires a certain flow rate for efficacious cell membrane perturbation. A flow rate that is too low may not adequately perturb the cell membranes. A flow rate that is too high may over-perturb and thus destroy the cell, rendering the sample unusable. In some embodiments, the cell perturbation filter(s) 110 may be configured to receive a volumetric flow rate between about 0.5-500 mL/min per mm² porous surface area. For example, the cell perturbation filter 110 may be configured to receive a volumetric flow rate of about 0.5-100, 0.5-10, 50-500, 5-100, or 5-20 mL/min per mm² porous surface area. In some embodiments, the cell perturbation filter 110 may be configured to receive a volumetric flow rate of greater than or equal to about 0.5, 1, 2, 5, 10, 12, 15, 18, 20, 25, 50, 75, 100, 125, 150, 175, 200, 250, 300, 350, 400, or 450 mL/min per mm² porous surface area. In some embodiments, the cell perturbation filter 110 may be configured to receive a volumetric flow rate of less than or equal to about 1, 2, 5, 10, 12, 15, 18, 20, 25, 50, 75, 100, 125, 150, 175, 200, 250, 300, 350, 400, 450, or 500 mL/min per mm² porous surface area. In some embodiments, the cell perturbation filter 110 may be configured to receive a volumetric flow rate greater than 500 mL/min per mm² porous surface area, such as about 550, 600, 650, 700, or 750 mL/min per mm² porous surface area. In some embodiments, the cell perturbation filter 110 may be configured to receive a volumetric flow rate less than 0.5 mL/min per mm² porous surface area, such as about 0.1, 0.2, 0.3, or 0.4 mL/min per mm² porous surface area.

The payload and the cell suspension may be mixed and passed through the one or more cell perturbation filters 110 for about 1 second. For example, the one or more pumps 114 may pump the cell suspension and the payload into and through mixer 108, and into and through the one or more cell perturbation filters 110 within less than or equal to about 0.1 s, 0.2 s, 0.5 s, 1 s, or 2 s.

The speed of the cells through the one or more cell perturbation filters 110 to induce cell membrane perturbation may be about 1 m/s. In some embodiments, the speed of the cells may be between 10 mm/s and 100 m/s. For example, the speed of the cells may be less than or equal to 10 mm/s, 20 mm/s, 50 mm/s, 1 m/s, 2 m/s, 5 m/s, 10 m/s, 20 m/s, 50 m/s, or 100 m/s. In some embodiments, the speed of the cells may be greater than or equal to 10 mm/s, 20 mm/s, 50 mm/s, 1 m/s, 2 m/s, 5 m/s, 10 m/s, 20 m/s, 50 m/s, or 100 m/s.

As mentioned above, the one or more cell perturbation filters 110 may be configured to deform the cell, causing a perturbation in the cell that allows the payload (e.g., an antigen) to enter the cell cytosol. The cell may be deformed from the one or more cell perturbation filters 110 for a period of time ranging from about 1 μs to at least about 10 ms, such as about 1 μs, 10 μs, 50 μs, 100 μs, 500 μs, 1 ms, 2 ms, 5 ms, or 10 ms. The perturbation in the cell can be defined as a breach in the cell that allows material from outside the cell to move into the cell (e.g., a hole, tear, cavity, aperture, pore, break, gap, perforation, etc.). The perturbation may be caused by pressure induced by mechanical strain and/or shear forces. The perturbation may be in the cell membrane. The perturbation may be transient. In some embodiments, the perturbation in the cell may persist for a duration between 1 second and 5 minutes. For example, the perturbation may persist for at least 1 s, 10 s, 30 s, 1 min, 2 min, 3 min, 4 min, 5 min, or more. In some embodiments, the perturbation may persist for less than 1 s, 10 s, 30 s, 1 min, 2 min, 3 min, 4 min, or 5 min.

The perturbed cell mixture produced from passing the mixture through the one or more cell perturbation filters 110 may be incubated for a duration of time. For example, the mixture may be incubated for about 0.0001 s to 20 min, such as 1 s, 30 s, 90 s, 2 min, 5 min, 10 min, 15 min, or 20 min. The mixture may be incubated at a temperature between 0° C. and 40° C., such as at least 0° C., 5° C., 10° C., 20° C., 25° C., 30° C., 35° C., or 40° C. The perturbed cell mixture may be incubated in one or more collection reservoirs 112, described in greater detail below.

Although not explicitly illustrated in the Figures, as described herein, it is contemplated that the payload and the cell suspension can be mixed after passage through the one or more cell perturbation filters 110. For example, the payload may be introduced to the perturbed cells in the cell suspension after the cell suspension is passed through the one or more filters 110. In this instance, the payload may be contained within tubing set 106 (e.g., and optionally housing 250) in a vessel (e.g., a payload reservoir 226 or mixer 108) fluidly connected to the one or more cell perturbation filters 110 to receive the perturbed cell suspension. In some embodiments, payload source 102 may be fluidly connected to a mixer 108 disposed subsequently to one or more cell perturbation filters 110.

Mechanoporation system 100 may comprise one or more collection reservoirs 112 fluidly connected to the one or more cell perturbation filters 110 to collect the perturbed cell mixture. The aforementioned one or more pumps 114 may be configured to move the perturbed cell mixture into the one or more collection reservoirs 112. Collection reservoirs 112 may comprise one or more flexible plastic bags, vials, vessels, or other containers configured to collect the perturbed cell mixture external to the tubing set 106. One or more collection reservoirs 112 may instead or additionally be disposed within tubing set 106. For example, FIGS. 2B, 2E, and 2F, illustrate example collection reservoirs 112 contained within housing 250 of tubing set 206.

The one or more collection reservoirs 112 may comprise a single collection reservoir 112. For example, FIGS. 2B and 2E illustrate one or more cell perturbation filters 110 fluidly connected to a single collection reservoir 112 to provide the perturbed cell mixture to the collection reservoir 112. The one or more collection reservoirs 112 may comprise a plurality of collection reservoirs 112. For example, as illustrated in FIG. 2F, tubing set 206F may comprise a plurality of cell perturbation filters 110, and each cell perturbation filter 110 may be fluidly connected to a given collection reservoir 112 of the plurality of collection reservoirs.

Once collected, the cell perturbed cell mixture may (as described above) be incubated (e.g., the cells may be frozen) for a period of time. The perturbed cell mixture may instead or additionally be washed prior to incubation. Finally, in combination with one or more of the aforementioned post-processing steps and/or immediately following collection, the perturbed cell mixture may be injected to a patient to execute the intended cell therapy with the cells.

Mechanoporation system 100 may have a throughput of less than or equal to 25 mL, 50 mL, 100 mL, 150 mL, 200 mL, 250 mL, 300 mL, or 400 mL of cell suspension per minute. In some embodiments, the throughput of mechanoporation system 100 may be greater than or equal to 50, 100, 150, 200, 250, 300, or 400 mL of cell suspension per minute.

The overall fluid throughput of system 100 may be less than or equal to 0.25 L, 0.5 L, 1 L, 1.5 L, 2 L, 2.5 L, or 5 L/min. In some embodiments, the overall fluid throughput may be greater than or equal to 0.25 L, 0.5 L, 1 L, 1.5 L, 2 L, 2.5 L, or 5 L/min.

Properties of the perturbed cell mixture, including cell viability, delivery efficiency, and cell retention can be tested using flow cytometry. In some examples, the tubing set 106 (or more generally, the mechanoporation system 100) may provide a perturbed cell mixture having a cell viability of at least 60%. For example, the perturbed cell mixture may have a cell viability of at least about 60%, 70%, 75%, 80%, 85%, 90%, 95%, 99%, or more. In some examples, the cell viability of the perturbed cell mixture may be between about 30-99%, 40-99%, 40-90%, 50-99%, 50-80%, 60-99%, 60-90%, or 60-80%.

In some examples, the delivery efficiency of the tubing set 106, quantified by the delivery of the payload into the cell, may be at least about 60%. For example, the delivery efficiency of the tubing set 106 may be about 60%, 70%, 75%, 80%, 85%, 90%, 95%, 99%, or more. In some examples, the delivery efficiency of the tubing set 106 may be between about 30-95%, 40-95%, 40-90%, 40-80%, 50-95%, 50-80%, 50-70%, 60-95%, or 60-80%.

The tubing set 106 (or mechanoporation system 100) may achieve each of the aforementioned cell viability and delivery efficiency in combination. In other words, the tubing set 106 may achieve a high delivery efficiency while maintaining a high cell viability of the sample. The tubing set 106 can achieve these properties because it is adaptable to the pressure and flow regimes set by an upstream pump or cell processing system.

The tubing set 106 may limit dead volume passed through the mechanoporation system. This property of the tubing set 106 can be evidenced by measuring the cell retention of the system. For example, the cell retention of the system may be at least about 60%. In some examples, the cell retention may be at least about 60%, 70%, 75%, 80%, 85%, 90%, 95%, or more.

The tubing set 106 may process large quantities of cells, for example, on the magnitude of millions, tens of millions, or hundreds of millions of cells. For example, the tubing set 106 may have a cell throughput of at least about 100 million, 150 million, 200 million, 225 million, 250 million, 275 million, 300 million, 325 million, 350 million, or a value therebetween. The cell throughput of the tubing set 206 may be related to the size of the cell perturbation filter(s) 110 therein. In one example, a relatively small cell perturbation filter having an overall filter surface area of 3.5 mm² may have a cell throughput of at least about 250 million cells. In another example, a larger cell perturbation filter having an overall filter surface area of 7 mm² may have a cell throughput of at least about 1 billion cells. Arranging several cell perturbation filters in parallel, as described herein, can increase the overall cell throughput of the mechanoporation system 100.

It is to be understood that term tubing set (e.g., tubing set 106) as used herein is not intended to be limited to flexible tubing and may instead or additionally include one or more fluidic channels and/or etchings in a surface of one or more rigid bodies (e.g., housing 250). Moreover, the processes of intracellular payload delivery as described herein are not intended to be limited to cell perturbation filters as described herein, and may instead or additionally include the use of standard mechanoporation devices known to one of ordinary skill in the art. For example, the one or more cell perturbation filters 110 may include microfluidic channels containing one or multiple constrictions in parallel or series, post configurations to induce perturbations, constrictions created through depth changes in fluidic channels, etc.

In some embodiments, a method for delivering a payload into cells may be provided. The method may comprise mixing a payload and a cell suspension to produce a mixture. For example, a mixer 108 fluidly connected to a payload source 102 comprising a payload and a cell source 104 comprising a cell suspension may mix the payload and the cell suspension to produce the mixture. As described herein, mixer 108 may be configured to contain the payload rather than being fluidly connected to an external payload source 102. Likewise, tubing set 106 may instead comprise a payload reservoir 226 fluidly connected to mixer 108 and configured to contain the payload. Mixer 108 may comprise in-line and static agitating geometry, an intersecting flow path (e.g., a Venturi mixer), and/or another mixing mechanism. Mixer 108 may be configured to mix the payload and the cell suspension without an agitation device or impeller. In some examples, the method may not include mixing the payload and the cell suspension. Rather, a cell and payload mixture 103 may be provided to the tubing set 106.

One or more pumps 114 may be configured to move the payload and the cell suspension into mixer 108. The one or more pumps 114 may additionally or alternatively move the cell and payload mixture 103 through the tubing set 106. The one or more pumps 114 may comprise a single pump including but not limited to a peristaltic pump or syringe pump. The one or more pumps 114 may be configured to provide a pressure gradient across the filter between 2 psi and 25 psi.

The method may include perturbing membranes of cells of the mixture as the mixture passes through one or more cell perturbation filters 110. The one or more cell perturbation filters 110 may be fluidly connected to mixer 108 to receive the mixture. In some examples, the one or more cell perturbation filters 110 may be fluidly connected to a cell processing system. One or more pumps 114 (e.g., of a cell processing system) may be configured to move the mixture through the one or more cell perturbation filters 110. The payload and the cell suspension may be mixed and passed through the one or more cell perturbation filters 110 for about 1 second. In some examples, passing the cell and payload mixture 103 (e.g., from a mixer 108 of the tubing set 106 or from an upstream cell processing system) to the one or more cell perturbation filters 110 may be prevented by one or more pressure regulators 116 configured to maintain an appropriate pressure within the tubing set 106 for efficacious mechanoporation. For example, the pressure regulator 116 may be a check valve that prevents flow of the cell and payload mixture 103 through the cell perturbation filter(s) 110 until a threshold pressure is reached in the tubing set 106. In some examples, pressure regulation may be controlled indirectly by controlling parameters of the upstream cell processing system. In this manner, the tubing set 106 may not require a pressure regulator.

In some embodiments, the payload and the cell suspension may be mixed after passing the cell suspension through the one or more cell perturbation filters 110. For example, the one or more pumps 114 may pass a cell suspension through the one or more cell perturbation filters 110 fluidly connected to cell source 104 and into mixer 108. Mixer 108 may be fluidly connected to the one or more cell perturbation filters 110 and may contain a payload, such that mixer 108 produces a perturbed cell mixture when mixer 108 receives the perturbed cell suspension from the one or more cell perturbation filters 110.

In some embodiments, the speed of the cells at the one or more cell perturbation filters 110 may be about 1 m/s. The one or more cell perturbation filters 110 may comprise a porous surface, such that as the cells in the mixture (or cell suspension) pass through pores in the porous surface, the cells can be deformed for a duration of time. Deforming the cells can cause the cell membranes to be perturbed. The payload may be delivered into the cell through the perturbations.

The method for delivering the payload into the cells may comprise collecting the perturbed cell mixture in one or more collection reservoirs 112. For example, one or more pumps 114 may be configured to move the mixture through the one or more cell perturbation filters 110 and into the one or more collection reservoirs 112.

In some embodiments, the aforementioned method may be performed at a temperature between about 0° C. and about 40° C. For example, the method may be carried out at room temperature (e.g., about 20° C.), physiological temperature (e.g., about 37° C.), higher than physiological temperature (e.g., about 37° C. or more), or at a reduced temperature (e.g., about 0° C. to 4° C.).

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EMBODIMENTS

Embodiment 1. A tubing set for delivering a payload into cells, the tubing set comprising:
   a cell source tubing portion configured to fluidly connect to a cell source comprising a cell suspension;
   a mixer fluidly connected to the cell source tubing portion and configured to mix the payload and the cell suspension to produce a mixture;
   a delivery portion comprising one or more cell perturbation filters fluidly connected to the mixer to receive the mixture and configured to perturb membranes of cells of the mixture as the mixture passes through the one or more cell perturbation filters; and
   a pump connection portion configured to connect to one or more pumps that are configured to move the cell suspension through the cell source tubing portion and into the mixer, to move the mixture from the mixer through the one or more cell perturbation filters, and to move a perturbed cell mixture into one or more collection reservoirs configured to collect the perturbed cell mixture.
Embodiment 2. The tubing set of embodiment 1, comprising a payload source tubing portion fluidly connected to the mixer and configured to fluidly connect to a payload source comprising the payload.
Embodiment 3. The tubing set of embodiment 2, wherein the one or more pumps are configured to move the payload through the payload source tubing portion and into the mixer.
Embodiment 4. The tubing set of embodiment 2 or embodiment 3, wherein the cell source tubing portion comprises a cell suspension pressure regulator and the payload source tubing portion comprises a payload pressure regulator.
Embodiment 5. The tubing set of embodiment 4, wherein one or more of the cell suspension pressure regulator or the payload pressure regulator comprises a check valve.
Embodiment 6. The tubing set of any one of embodiments 2-5, wherein the payload source is fluidly connected to the cell source to receive the cell suspension from the cell source.
Embodiment 7. The tubing set of embodiment 6, comprising a single pressure regulator fluidly connected to the payload source to receive the cell suspension and the payload.
Embodiment 8. The tubing set of any one of embodiments 1-7, wherein the mixer is configured to normalize pulsations from the one or more pumps.
Embodiment 9. The tubing set of any one of embodiments 1-8, comprising a mixture pressure regulator fluidly connected to the mixer to receive the mixture.
Embodiment 10. The tubing set of any one of embodiments 1-9, wherein the mixer comprises an intersecting flow path, in-line agitating geometry, or a Venturi mixer in the tubing set.
Embodiment 11. The tubing set of any one of embodiments 1-10, wherein the mixer is configured to mix the payload and the cell suspension without an agitation device or impeller.
Embodiment 12. The tubing set of any one of embodiments 1-11, wherein each filter of the one or more cell perturbation filters comprises a porous surface, each pore of the porous surface comprising a diameter between 1 μm and 10 μm.
Embodiment 13. The tubing set of embodiment 12, wherein the porous surface comprises uniformly-sized pores.
Embodiment 14. The tubing set of embodiment 12 or embodiment 13, wherein the diameter of the pores is between 10% and 70% of a diameter of the cells.
Embodiment 15. The tubing set of any one of embodiments 1-14, wherein the one or more cell perturbation filters comprises a thickness between 0.1 μm and 1 mm.
Embodiment 16. The tubing set of any one of embodiments 1-15, wherein the one or more cell perturbation filters comprises a plurality of cell perturbation filters.
Embodiment 17. The tubing set of embodiment 16, wherein the plurality of cell perturbation filters are disposed in the tubing set in parallel to one another, such that each filter of the plurality of cell perturbation filters is fluidly connected to the mixer and is configured to be fluidly connected to the one or more collection reservoirs.
Embodiment 18. The tubing set of embodiment 17, wherein the one or more collection reservoirs comprises a single collection reservoir.
Embodiment 19. The tubing set of embodiment 17 or embodiment 18, wherein the one or more collection reservoirs comprises a plurality of collection reservoirs, each collection reservoir of the plurality of collection reservoirs configured to be fluidly connected to a given filter of the plurality of cell perturbation filters.
Embodiment 20. The tubing set of any one of embodiments 1-19, wherein the one or more cell perturbation filters comprises silicon nitride, polycarbonate, or another polymer.
Embodiment 21. The tubing set of any one of embodiments 1-20, comprising one or more leukocyte reduction filters or cell strainers.
Embodiment 22. The tubing set of embodiment 21, wherein the one or more leukocyte reduction filters or cell strainers are fluidly connected to the cell source to receive the cell suspension.
Embodiment 23. The tubing set of embodiment 21 or embodiment 22, wherein the cell source tubing portion comprises the one or more leukocyte reduction filters or cell strainers.
Embodiment 24. The tubing set of any one of embodiments 1-23, comprising a collection reservoir tubing portion fluidly connected to the delivery portion and configured to fluidly connect to the one or more collection reservoirs.
Embodiment 25. The tubing set of any one of embodiments 1-24, comprising one or more of polyvinyl chloride tubing or C-flex tubing.
Embodiment 26. The tubing set of any one of embodiments 1-25, comprising a housing containing at least the mixer and the one or more cell perturbation filters.
Embodiment 27. The tubing set of embodiment 26, wherein the housing is configured to be removably connected to the tubing set.
Embodiment 28. The tubing set of embodiment 26 or embodiment 27, wherein the housing contains the one or more collection reservoirs.
Embodiment 29. The tubing set of any one of embodiments 26-28, wherein the housing comprises a payload reservoir configured to contain the payload.
Embodiment 30. The tubing set of embodiment 29, wherein the tubing set is provided with the payload stored in the payload reservoir.
Embodiment 31. The tubing set of embodiment 29 or embodiment 30, wherein the payload reservoir is configured to normalize pulsations from the one or more pumps.
Embodiment 32. The tubing set of any one of embodiments 1-31, wherein the mixer is configured to contain the payload.
Embodiment 33. The tubing set of embodiment 32, wherein the tubing set is provided with the payload stored in the mixer.
Embodiment 34. The tubing set of embodiment 32 or embodiment 33, wherein the mixer is configured to normalize pulsations from the one or more pumps.
Embodiment 35. The tubing set of any one of embodiments 1-34, wherein the delivery portion is configured to receive a volume between at least 1 mL and 500 mL.
Embodiment 36. The tubing set of any one of embodiments 1-35, wherein the delivery portion is configured to receive a volume between at most 500 mL and 5 L.
Embodiment 37. The tubing set of any one of embodiments 1-36, wherein the one or more pumps comprises a single pump.
Embodiment 38. The tubing set of embodiment 37, wherein the pump comprises a peristaltic pump or syringe pump.
Embodiment 39. The tubing set of any one of embodiments 1-38, comprising a pulsation damper configured to normalize pulsations from the one or more pumps.
Embodiment 40. The tubing set of embodiment 39, wherein the pulsation damper is fluidly connected to the mixer to receive the mixture.
Embodiment 41. The tubing set of any one of embodiments 1-40, wherein the tubing set is configured to fluidly connect to one or more of the cell source, a payload source, and the one or more collection reservoirs using tube welded or swaged connections.
Embodiment 42. The tubing set of any one of embodiments 1-41, wherein the tubing set is configured to fluidly connect to one or more of the cell source, a payload source, and the one or more collection reservoirs using one or more connectors.
Embodiment 43. The tubing set of embodiment 42, wherein the one or more connectors comprise one or more Luer connectors, threaded connectors, or quick disconnect connectors.
Embodiment 44. The tubing set of any one of embodiments 1-43, wherein the tubing set does not comprise flow sensors or pressure sensors.
Embodiment 45. A mechanoporation system for delivering a payload into cells, the system comprising:
   a payload source comprising the payload;
   a cell source comprising a cell suspension;
   a tubing set comprising:
      a mixer fluidly connected to the payload source and to the cell source and configured to mix the payload and the cell suspension to produce a mixture; and
      one or more cell perturbation filters fluidly connected to the mixer to receive the mixture and configured to perturb membranes of cells of the mixture as the mixture passes through the one or more cell perturbation filters;
   one or more collection reservoirs fluidly connected to the one or more cell perturbation filters to collect the perturbed cell mixture; and
   one or more pumps connected to the tubing set and configured to move the payload and the cell suspension into the mixer, to move the mixture from the mixer through the one or more cell perturbation filters, and to move the perturbed cell mixture into the one or more collection reservoirs.
Embodiment 46. The system of embodiment 45, wherein the payload source comprises a flexible bag or vial comprising the payload.
Embodiment 47. The system of embodiment 45 or embodiment 46, wherein the cell source comprises a flexible bag or vial comprising the cell suspension.
Embodiment 48. The system of any one of embodiments 45-47, comprising a plurality of pressure regulators including a payload pressure regulator and a cell suspension pressure regulator, the cell suspension pressure regulator fluidly connected to the cell source, and the payload pressure regulator fluidly connected to the payload source.
Embodiment 49. The system of embodiment 48, wherein one or more pressure regulators of the plurality of pressure regulators comprises a check valve.
Embodiment 50. The system of any one of embodiments 45-49, wherein the payload source is fluidly connected to the cell source to receive the cell suspension from the cell source.
Embodiment 51. The system of embodiment 50, comprising a single pressure regulator fluidly connected to the payload source to receive the cell suspension and the payload.
Embodiment 52. The system of any one of embodiments 45-51, wherein the one or more pumps comprises a single pump.
Embodiment 53. The system of embodiment 52, wherein the pump comprises a peristaltic pump or syringe pump.
Embodiment 54. The system of any one of embodiments 45-53, wherein a speed of a cell in the cells of the mixture as the mixture passes through the one or more cell perturbation filters is between 10 mm/s and 100 m/s.
Embodiment 55. The system of any one of embodiments 45-54, wherein the mixer is configured to normalize pulsations from the one or more pumps.
Embodiment 56. The system of any one of embodiments 45-55, comprising a mixture pressure regulator fluidly connected to the mixer to receive the mixture.
Embodiment 57. The system of any one of embodiments 45-56, wherein the mixer comprises an intersecting flow path, in-line agitating geometry, or a Venturi mixer in the tubing set.
Embodiment 58. The system of any one of embodiments 45-57, wherein the mixer is configured to mix the payload and the cell suspension without an agitation device or impeller.
Embodiment 59. The system of any one of embodiments 45-58, wherein the tubing set comprises a pulsation damper configured to normalize pulsations from the one or more pumps.
Embodiment 60. The system of embodiment 59, wherein the pulsation damper is fluidly connected to the mixer to receive the mixture.
Embodiment 61. The system of any one of embodiments 45-60, wherein each filter of the one or more cell perturbation filters comprises a porous surface, each pore of the porous surface comprising a diameter between 1 μm and 10 μm.
Embodiment 62. The system of embodiment 61, wherein the porous surface comprises uniformly-sized pores.
Embodiment 63. The system of embodiment 61 or embodiment 62, wherein the diameter of the pores is between 10% and 70% of a diameter of the cells.
Embodiment 64. The system of any one of embodiments 45-63, wherein the one or more cell perturbation filters comprises a thickness between 0.1 μm and 1 mm.
Embodiment 65. The system of any one of embodiments 45-64, wherein the one or more cell perturbation filters comprises a plurality of cell perturbation filters.
Embodiment 66. The system of embodiment 65, wherein the plurality of cell perturbation filters are disposed in the tubing set in parallel to one another, such that each filter of the plurality of cell perturbation filters is fluidly connected to the mixer and to the one or more collection reservoirs.
Embodiment 67. The system of embodiment 66, wherein the one or more collection reservoirs comprises a single collection reservoir.
Embodiment 68. The system of embodiment 66 or embodiment 67, wherein the one or more collection reservoirs comprises a plurality of collection reservoirs, each collection reservoir of the plurality of collection reservoirs fluidly connected to a given filter of the plurality of cell perturbation filters.
Embodiment 69. The system of any one of embodiments 45-68, wherein the one or more cell perturbation filters comprises silicon nitride, polycarbonate, or another polymer.
Embodiment 70. The system of any one of embodiments 45-69, wherein the tubing set comprises one or more leukocyte reduction filters or cell strainers.
Embodiment 71. The system of embodiment 70, wherein the one or more leukocyte reduction filters or cell strainers are fluidly connected to the cell source to receive the cell suspension.
Embodiment 72. The system of embodiment 70 or embodiment 71, wherein the one or more leukocyte reduction filters or cell strainers are fluidly connected to the cell source to receive the cell suspension.
Embodiment 73. The system of any one of embodiments 45-72, wherein the one or more collection reservoirs are disposed within the tubing set.
Embodiment 74. The system of any one of embodiments 45-73, wherein the one or more collection reservoirs comprises one or more flexible bags, vials, or containers configured to contain the perturbed cell mixture.
Embodiment 75. The system of any one of embodiments 45-74, wherein the tubing set comprises one or more of polyvinyl chloride tubing or C-flex tubing.
Embodiment 76. The system of any one of embodiments 45-75, wherein the tubing set comprises a housing containing at least the mixer and the one or more cell perturbation filters.
Embodiment 77. The system of embodiment 76, wherein the housing is configured to be removably connected to the tubing set.
Embodiment 78. The system of embodiment 76 or embodiment 77, wherein the housing contains the one or more collection reservoirs.
Embodiment 79. The system of any one of embodiments 45-78, wherein the tubing set is fluidly connected to one or more of the payload source, the cell source, and the one or more collection reservoirs using tube welded or swaged connections.
Embodiment 80. The system of any one of embodiments 45-79, comprising one or more connectors configured to fluidly connect the tubing set to one or more of the payload source, the cell source, and the one or more collection reservoirs.
Embodiment 81. The system of embodiment 80, wherein the one or more connectors comprise one or more Luer connectors, threaded connectors, or quick disconnect connectors.
Embodiment 82. The system of any one of embodiments 45-81, wherein the tubing set does not comprise an external power source.
Embodiment 83. The system of any one of embodiments 45-82, wherein the tubing set does not comprise flow sensors or pressure sensors.
Embodiment 84. A method for delivering a payload into cells, the method comprising:
   mixing, by a mixer fluidly connected to a payload source comprising a payload and to a cell source comprising a cell suspension, the payload and the cell suspension to produce a mixture;
   perturbing, by one or more cell perturbation filters fluidly connected to the mixer to receive the mixture, membranes of cells of the mixture as the mixture passes through the one or more cell perturbation filters; and
   collecting, by one or more collection reservoirs fluidly connected to the one or more cell perturbation filters, the perturbed cell mixture,
   wherein one or more pumps are configured to move the payload and the cell suspension into the mixer, to move the mixture from the mixer through the one or more cell perturbation filters, and to move the perturbed cell mixture into the one or more collection reservoirs.
Embodiment 85. The method of embodiment 84, wherein the payload source comprises a flexible bag or vial comprising the payload.
Embodiment 86. The method of embodiment 84 or embodiment 85, wherein the cell source comprises a flexible bag or vial comprising the cell suspension.
Embodiment 87. The method of any one of embodiments 84-86, wherein the payload source is fluidly connected to the cell source to receive the cell suspension from the cell source.
Embodiment 88. The method of any one of embodiments 84-87, wherein the one or more pumps comprises a single pump.
Embodiment 89. The method of embodiment 88, wherein the pump comprises a peristaltic pump or syringe pump.
Embodiment 90. The method of any one of embodiments 84-89, wherein a speed of a cell in the cells of the mixture as the mixture passes through the one or more cell perturbation filters is between 10 mm/s and 100 m/s.
Embodiment 91. The method of any one of embodiments 84-90, wherein the mixer comprises one or more tubing portions comprising an intersecting flow path, in-line agitating geometry, or a Venturi mixer.
Embodiment 92. The method of any one of embodiments 84-91, comprising mixing the payload and the cell suspension without an agitation device or impeller.
Embodiment 93. The method of any one of embodiments 84-92, comprising a pulsation damper configured to normalize pulsations from the one or more pumps.
Embodiment 94. The method of embodiment 93, wherein the pulsation damper is fluidly connected to the mixer to receive the mixture.
Embodiment 95. The method of any one of embodiments 84-94, comprising a plurality of pressure regulators including a cell suspension pressure regulator fluidly connected to the cell source and a payload pressure regulator fluidly connected to the payload source, wherein one or more pressure regulators of the plurality of the pressure regulators comprise a check valve.
Embodiment 96. The method of any one of embodiments 84-95, wherein each filter of the one or more cell perturbation filters comprises a porous surface, each pore of the porous surface comprising a diameter between 1 μm and 10 μm.
Embodiment 97. The method of embodiment 96, wherein the porous surface comprises uniformly-sized pores.
Embodiment 98. The method of embodiment 96 or embodiment 97, wherein the diameter of the pores is between 10% and 70% of a diameter of the cells.
Embodiment 99. The method of any one of embodiments 84-98, wherein the one or more cell perturbation filters comprises a thickness between 0.1 μm and 1 mm.
Embodiment 100. The method of any one of embodiments 84-99, wherein the one or more cell perturbation filters comprises a plurality of cell perturbation filters.
Embodiment 101. The method of embodiment 100, wherein the plurality of cell perturbation filters are disposed in the tubing set in parallel to one another, such that each filter of the plurality of cell perturbation filters is fluidly connected to the mixer and to the one or more collection reservoirs.
Embodiment 102. The method of embodiment 101, wherein the one or more collection reservoirs comprises a single collection reservoir.
Embodiment 103. The method of embodiment 101 or embodiment 102, wherein the one or more collection reservoirs comprises a plurality of collection reservoirs, each collection reservoir of the plurality of collection reservoirs fluidly connected to a given filter of the plurality of cell perturbation filters.
Embodiment 104. The method of any one of embodiments 84-103, comprising one or more leukocyte reduction filters or cell strainers.
Embodiment 105. The method of embodiment 104, wherein the one or more leukocyte reduction filters or cell strainers are fluidly connected to the cell source to receive the cell suspension.
Embodiment 106. The method of embodiment 104 or embodiment 105, wherein the one or more leukocyte reduction filters or cell strainers are fluidly connected to the cell source to receive the cell suspension.
Embodiment 107. The method of any one of embodiments 84-106, wherein the one or more collection reservoirs comprises one or more flexible bags, vials, or containers configured to contain the perturbed cell mixture.
Embodiment 108. The method of any one of embodiments 84-107, comprising a housing containing at least the mixer and the one or more cell perturbation filters.
Embodiment 109. The method of embodiment 108, wherein the housing contains the one or more collection reservoirs.
Embodiment 110. A tubing set for delivering a payload into cells, the tubing set comprising:
   a cell source tubing portion configured to fluidly connect to a cell source comprising a cell suspension;
   a delivery portion comprising one or more cell perturbation filters fluidly connected to the cell source tubing portion to receive the cell suspension and configured to perturb membranes of cells of the cell suspension as the cell suspension passes through the one or more cell perturbation filters;
   a mixer fluidly connected to the delivery portion and configured to mix a payload and a perturbed cell suspension to produce a perturbed cell mixture; and
   a pump connection portion configured to connect to one or more pumps that are configured to move the cell suspension through the cell source tubing portion and through the one or more cell perturbation filters, to move the perturbed cell suspension from the delivery portion and into the mixer, and to move the perturbed cell mixture into one or more collection reservoirs configured to collect the perturbed cell mixture.
Embodiment 111. The tubing set of any one of embodiments 1-44 or 110, wherein the tubing set is configured to provide the perturbed cell mixture having a cell viability of at least 60%.
Embodiment 112. The tubing set of any one of embodiments 1-44, 110, or 111, wherein a delivery efficiency of the tubing set in perturbing the membranes of the cells is at least 60%.
Embodiment 113. The tubing set of any one of embodiments 1-44, or 110-112, wherein the one or more cell perturbation filters comprises silicon, silicon nitride, silicon oxide, polycarbonate, or another polymer.
Embodiment 114. The tubing set of any one of embodiments 1-11, 13-44, or 110-113, wherein each filter of the one or more cell perturbation filters comprises a porous surface, each pore of the porous surface comprising a diameter between 1 μm and 20 μm.
Embodiment 115. The tubing set of any one of embodiments 12, 13, 110, or 114 wherein the diameter of the pores is between 10% and 90% of a diameter of the cells.
Embodiment 116. The system of any one of embodiments 45-83, wherein the tubing set is configured to provide the perturbed cell mixture having a cell viability of at least 60%.
Embodiment 117. The system of any one of embodiments 45-83 or 116, wherein a delivery efficiency of the tubing set in perturbing the membranes of the cells is at least 60%.
Embodiment 118. The system of any one of embodiments 45-83, 116, or 117, wherein the one or more cell perturbation filters comprises silicon, silicon nitride, silicon oxide, polycarbonate, or another polymer.
Embodiment 119. The system of any one of embodiments 45-60, 62-83, or 116-118, wherein each filter of the one or more cell perturbation filters comprises a porous surface, each pore of the porous surface comprising a diameter between 1 μm and 20 μm.
Embodiment 120. The system of any one of embodiments 61, 62, or 119 wherein the diameter of the pores is between 10% and 90% of a diameter of the cells.
Embodiment 121. The method of any one of embodiments 84-110, wherein the tubing set provides the perturbed cell mixture having a cell viability of at least 60%.
Embodiment 122. The method of any one of embodiments 84-110 or 121, wherein a delivery efficiency of the tubing set in perturbing the membranes of the cells is at least 60%.
Embodiment 123. The method of any one of embodiments 84-110, 121, or 122, wherein the one or more cell perturbation filters comprises silicon, silicon nitride, silicon oxide, polycarbonate, or another polymer.
Embodiment 124. The method of any one of embodiments 84-95, 97-110, or 121-123, wherein each filter of the one or more cell perturbation filters comprises a porous surface, each pore of the porous surface comprising a diameter between 1 μm and 20 μm.
Embodiment 125. The method of any one of embodiments 96, 97, or 124, wherein the diameter of the pores is between 10% and 90% of a diameter of the cells.

### EXAMPLES

Experimentation demonstrating various tubing set setups that have been integrated with various existing cell processing systems was conducted and is described below. In each of the following examples, the tubing sets were fluidly connected to a cell processing system (e.g., Thermo Fisher Rotea™ or Fresenius Kabi Lovo®, specified below) to receive a cell mixture including a cell suspension and a payload from the cell processing system. The various components of the tubing sets were fluidly connected to the upstream cell processing system using tubing having an inner diameter of about 3.18 mm (⅛ inch) and an outer diameter of about 6.35 mm (¼ inch) or about 8 mm ( 5/16 inch). These tubing dimensions can be easily adapted for tube weld and Luer lock connections.

The payload used in each of the example experiments was Dextran. The concentration and size of the Dextran in the payload was 0.1 mg/mL and 3 kDa, respectively. Dextran is a fluorescently labeled sugar polymer that can be a proxy for payloads such as mRNA, CRISPR RNP, etc. because it has similar delivery behavior. In each of the following examples, the concentration of the cell mixture was 20×10⁶ cells/mL. Prior to passing the cell mixture through the tubing set, each of the systems (including the tubing and components therein) was primed with a cell media solution (e.g., Opti-Mem media). 10 mL of the perturbed cell mixture was collected in a collection reservoir (e.g., a tube) to evaluate cell viability and Dextran delivery (i.e., delivery efficiency) using flow cytometry.

### Example 1

To perform the experiment, a Leukopak sample of peripheral blood mononuclear cells (PBMCs) was retrieved and the PBMCs were first isolated from the sample using Ficoll separation. The isolated PBMCs were provided to a Thermo Fisher Rotea™ cell processing system in which they were mixed with a payload (Dextran) to produce a cell mixture. Rotea™ is understood to be representative of cell processing systems that include a peristaltic pump and/or centrifuge mechanism.

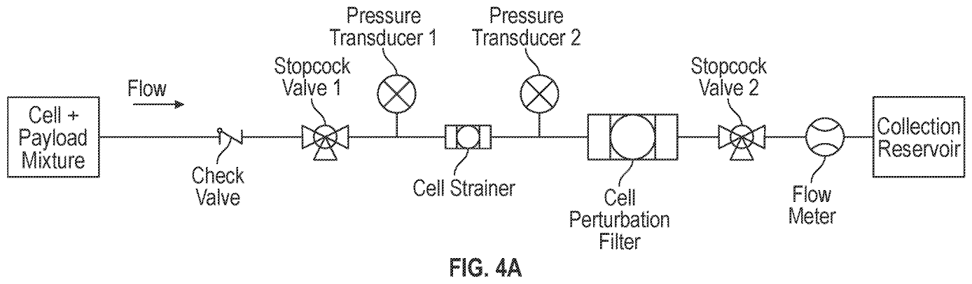

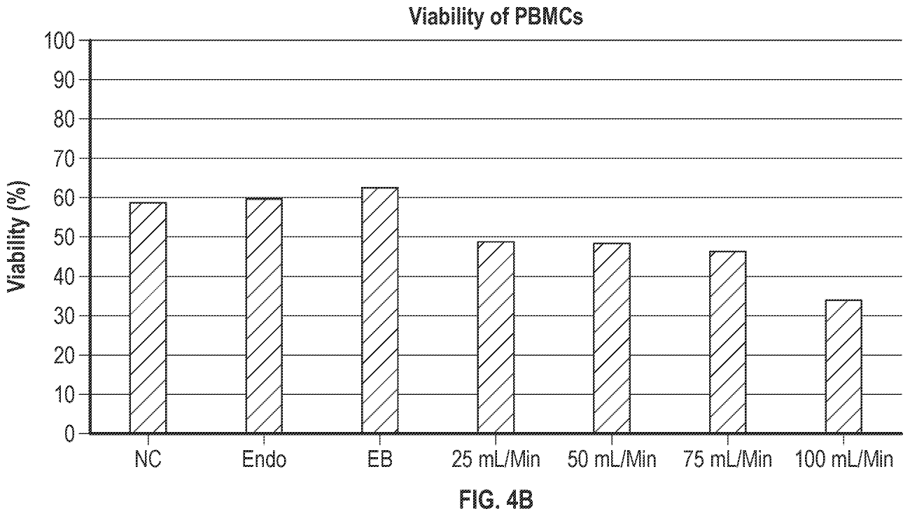

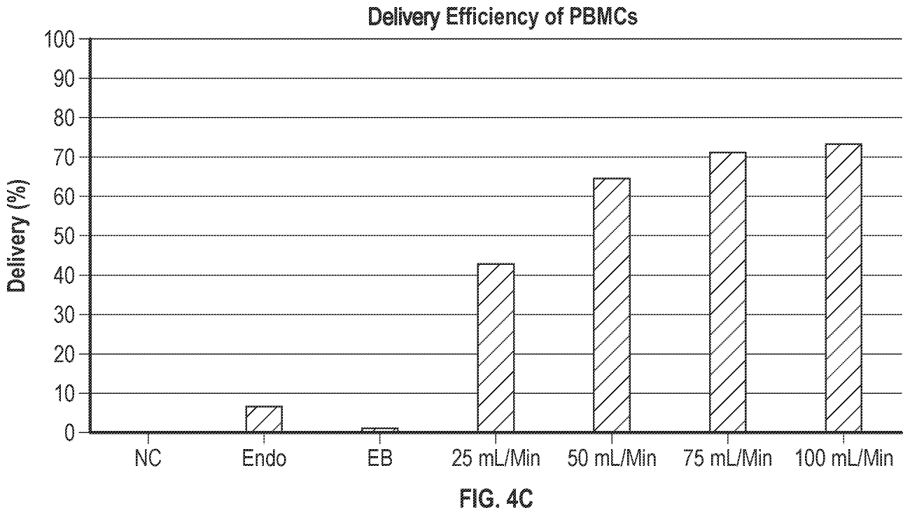

FIG. 4A illustrates the setup of the Example 1 tubing set. The cell perturbation filter used in Example 1 was a 13 mm² polymer filter (or membrane) having 5 μm pores, although, as described herein, tubing sets are not limited to this type of filter. The setup includes a check valve (pressure regulator) fluidly connected to the cell mixture source (i.e., the cell processing system) that ensures the cell mixture is at a pressure adequate for perturbing cell membranes prior to passing through the tubing set. The setup also includes a or cell strainer for removing larger unwanted particulates in the cell mixture prior to passing the mixture through the cell perturbation filter. The pore size of the cell strainer in this setup is 105 μm. As each of the experimental setups described herein were designed to evaluate tubing sets when integrated with cell processing systems, the tubing set also includes several components that facilitate evaluating the system during use, including stopcock valves, pressure transducers, and a flow meter. It is to be understood that, in practice, a tubing set integrated to a cell processing system may not require one or more of the aforementioned components.

The samples tested included four samples that were passed through the tubing set at different rates-25 mL/min, 50 mL/min, 75 mL/min, and 100 mL/min. Three control samples were also tested, including a no contact (“NC”) sample, endocytosis (“endo”) sample, and empty boost (“EB”) sample. The NC sample is a sample in which no payload was introduced to the cells, and the cells were not passed through the tubing set. The endo sample is a sample in which the cells were exposed to the payload, but the cell mixture was not passed through the tubing set. The EB sample is a sample in which no payload was introduced to the cells, but the cells were passed through the tubing set.

The results of testing the Example 1 tubing set (illustrated in FIG. 4A) integrated with the Rotea™ cell processing system are provided in Tables 1A-1B and FIGS. 4B-4C. The graphs illustrated in FIGS. 4B-4C correspond to the data presented in Table 1A. As noted above, for each sample, the cell viability (Table 1A, FIG. 4B) and delivery efficiency (Table 1A, FIG. 4C) were measured. The estimated cell throughput was also measured and is provided below in Table 1B.

TABLE 1A
Viability and Delivery Efficiency of PBMCs

| | Viability | Delivery |
|---|---|---|
| Test | (%) | (%) |
| NC | 59.1 | 0.22 |
| Endo | 60.1 | 6.45 |
| EB | 63 | 1.13 |

| | | | |
|---|---|---|---|
| 25 | mL/min | 49.2 | 43 |
| 50 | mL/min | 48.8 | 64.6 |
| 75 | mL/min | 46.6 | 71.1 |
| 100 | mL/min | 34.2 | 73.4 |

TABLE 1B
Estimated Throughput of PBMCs

| | Throughput |
|---|---|
| Test | (millions of cells) |

| | | |
|---|---|---|
| 25 | mL/min | 208 |
| 50 | mL/min | 333 |
| 75 | mL/min | 325 |
| 100 | mL/min | 300 |

The cell viability of PBMCs passed through the Example 1 tubing set at each of the flow rates 25 mL/min, 50 mL/min, and 75 mL/min was about the same at about 50%. The flow rate of 100 mL/min produced the lowest cell viability at about 35%. The delivery efficiency of the tubing set, quantified by measuring the delivery of the payload (Dextran) in the cells, increased as the flow rate was increased. At the lowest tested flow rate of 25 mL/min, the delivery efficiency was 43%. At the highest flow rate, the delivery efficiency was about 73.4%. The Example 1 tubing set was capable of processing over 200 million cells in one pass, and at flow rates greater than (and equal to) 50 mL/min, over 300 million cells in one pass. These results demonstrate the ability of the tubing sets described herein to process large quantities of cells.

### Example 2

Similar to the experiment in Example 1, a Leukopak sample of PBMCs was retrieved and the PBMCs were first isolated from the sample using Ficoll separation. The isolated PBMCs were provided to a Thermo Fisher Rotea™ cell processing system in which they were mixed with a payload (Dextran) to produce a cell mixture.

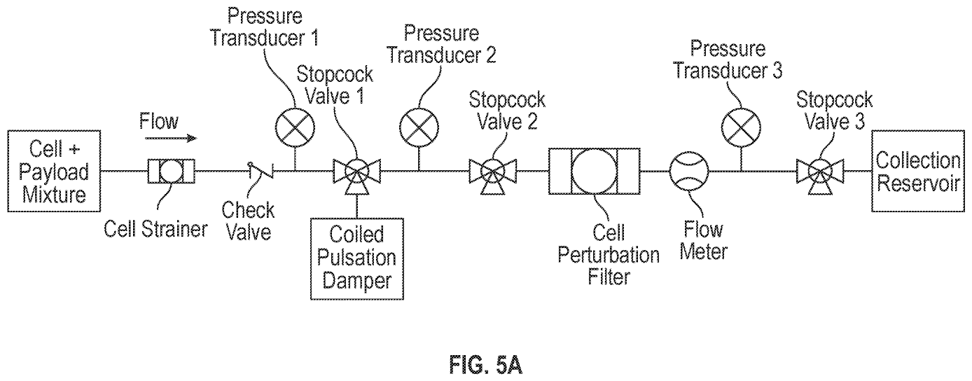

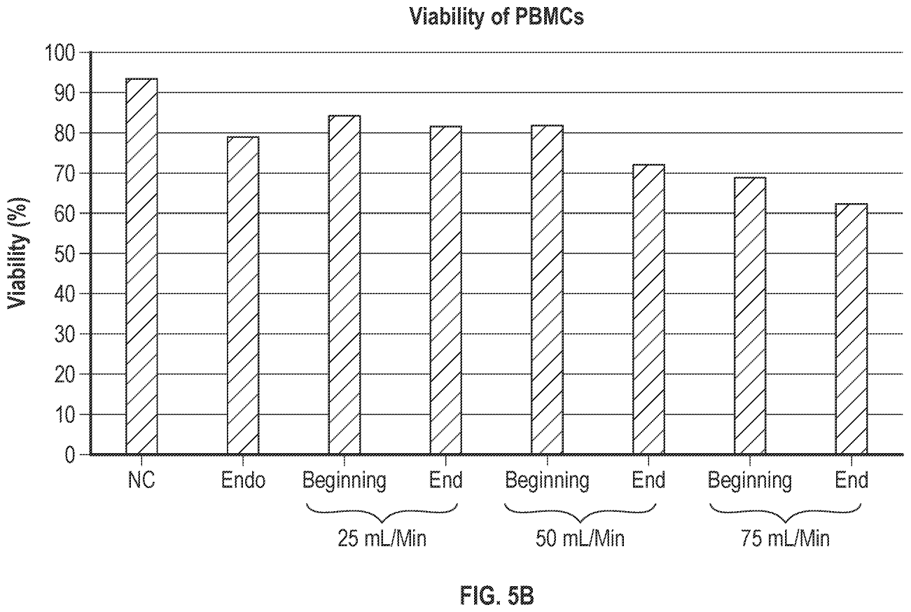

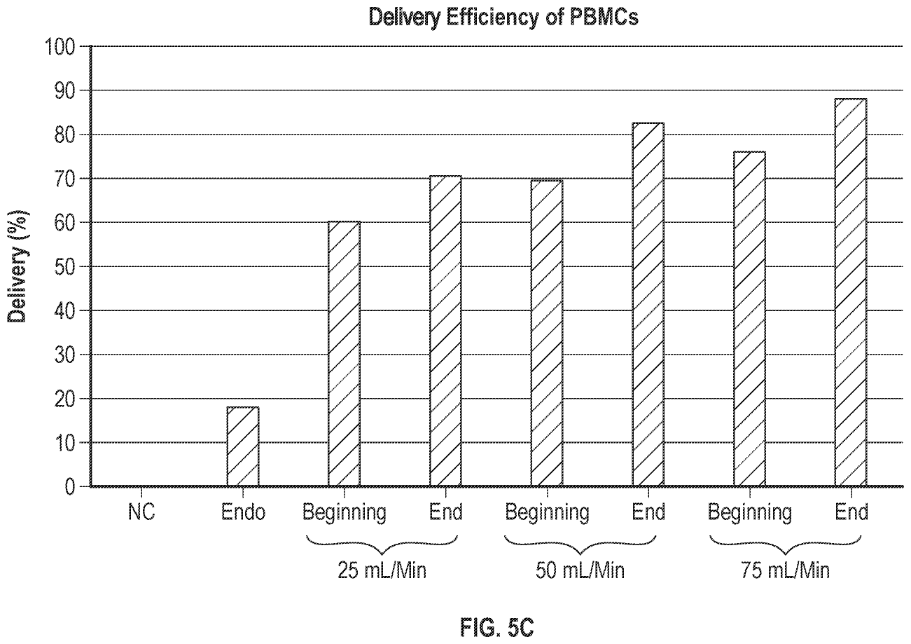

FIG. 5A illustrates the setup of the Example 2 tubing set. The cell perturbation filter used in Example 2 was a 13 mm² polymer filter (or membrane) having 5 μm pores. The setup includes a check valve (pressure regulator) fluidly connected to the cell mixture source (i.e., the cell processing system) that ensures the cell mixture is at a pressure adequate for perturbing cell membranes prior to passing through the tubing set. The setup also includes a cell strainer upstream of the check valve for removing larger unwanted particulates in the cell mixture prior to passing the mixture through the cell perturbation filter. The pore size of the cell strainer in this setup is 105 μm. The setup of Example 2 also includes a pulsation damper for reducing pressure oscillations introduced to the tubing set by the upstream cell processing system. The pulsation damper is flexible tubing arranged as coil that is 500 mm in length. The pulsation damper may be optional in tubing sets, for example, dependent on the cell processing system that the tubing set is integrated to. Some cell processing systems may introduce larger pressure oscillations that necessitate a damper, whereas other cell processing systems may exhibit only minimal pressure oscillations and thus may not necessitate a damper. As noted above in Example 1, the other illustrated components in the tubing set in FIG. 5A (e.g., pressure transducers, stopcock valves, and/or flow meters) are for testing the tubing set. One or more of these components may be eliminated from the tubing set when integrating it to a cell processing system for clinical/research use.

The samples tested included three samples that were passed through the tubing set at different rates—25 mL/min, 50 mL/min and 75 mL/min. Two control samples were also tested, including a no contact (“NC”) sample and endocytosis (“endo”) sample (each of which are described above in Example 1).

The results of testing the Example 2 tubing set (illustrated in FIG. 5A) integrated with the Rotea™ cell processing system are provided in Tables 2A-2B and FIGS. 5B-5C. The graphs illustrated in FIGS. 5B-5C correspond to the data presented in Table 2A. For each sample, the cell viability (Table 2A, FIG. 5B) and delivery efficiency (Table 2A, FIG. 5C) were measured at the beginning and at the end of collection. The estimated cell throughput was also measured and is provided below in Table 2B.

TABLE 2A
Viability and Delivery Efficiency of PBMCs

| | Viability | Delivery |
|---|---|---|
| Test | (%) | (%) |

| NC | | | 93.5 | 0 |
|---|---|---|---|---|
| Endo | | | 78.9 | 18 |
| | 25 | Beginning | 84.4 | 60.3 |
| | mL/min | End | 81.5 | 70.8 |
| | 50 | Beginning | 81.9 | 69.5 |
| | mL/min | End | 72.3 | 82.7 |
| | 75 | Beginning | 68.8 | 76.1 |
| | mL/min | End | 62.5 | 88 |

TABLE 2B
Estimated Throughput of PBMCs

| | Throughput |
|---|---|
| Test | (millions of cells) |
| 25 mL/min | 225 |
| 50 mL/min | 217 |

The cell viability of PBMCs passed through the Example 2 tubing set was above 60% for each of the flow rates 25 mL/min, 50 mL/min, and 75 mL/min. The flow rate of 25 mL/min exhibited the highest cell viability at above 80% (including the initial and final readout). The flow rate of 75 mL/min exhibited the lowest cell viability at just above 60% (at final readout). Within each flow rate, there was less than 10% of variance between the initial viability measurement and final viability measurement. The flow rate of 25 mL/min exhibited the smallest amount of variance at about 3%. The flow rate of 50 mL/min exhibited the largest amount of variance at about 10%. The delivery efficiency of the Example 2 tubing set, quantified by measuring the delivery of the payload (Dextran) in the cells, generally increased as the flow rate was increased. Within each tested flow rate, the final delivery efficiency measured was greater than the initial delivery efficiency measured, with a difference of about 10% between the initial and final readouts in each flow rate. At the flow rate of 25 mL/min, the final delivery efficiency was 70%. At the highest flow rate, the delivery efficiency was close to 90% at about 88%. The Example 2 tubing set was capable of processing over 200 million cells in one pass. As stated above with respect to the Example 1 tubing set, these results demonstrate the ability of the tubing sets described herein to process large quantities of cells.

### Example 3

Similar to the above examples, to perform the experiment, a Leukopak sample of PBMCs was retrieved and the PBMCs were first isolated from the sample using Ficoll separation. However, Example 3 differs from Examples 1 and 2 above because after isolation, the isolated PBMCs were provided to a Fresenius Kabi Lovo™ cell processing system in which they were mixed with a payload (Dextran) to produce a cell mixture. Lovo™ is understood to be representative of cell processing systems that operate using a peristaltic pump.

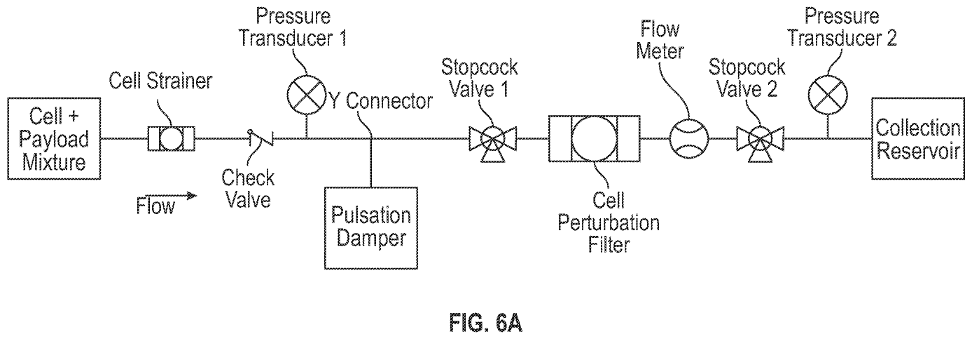

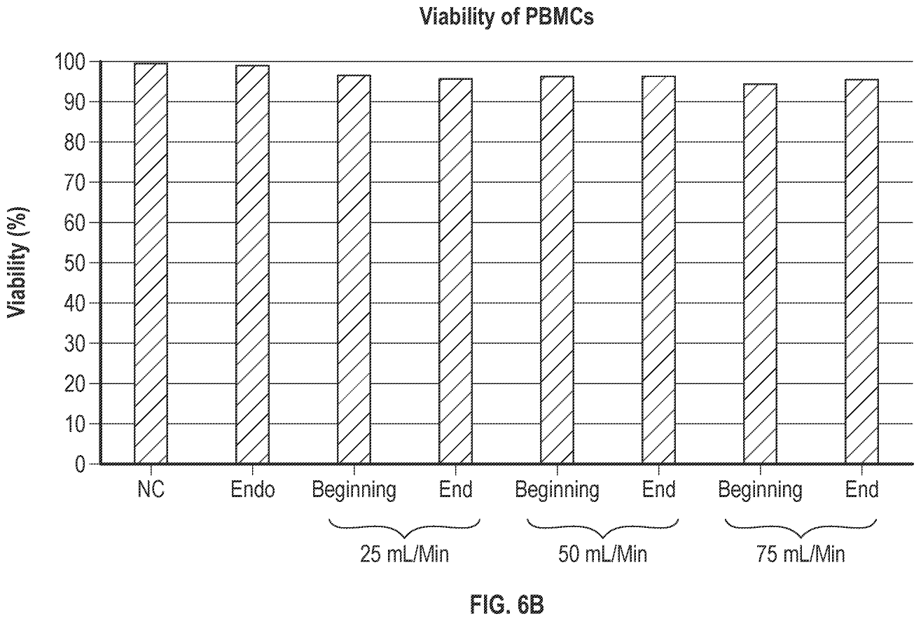

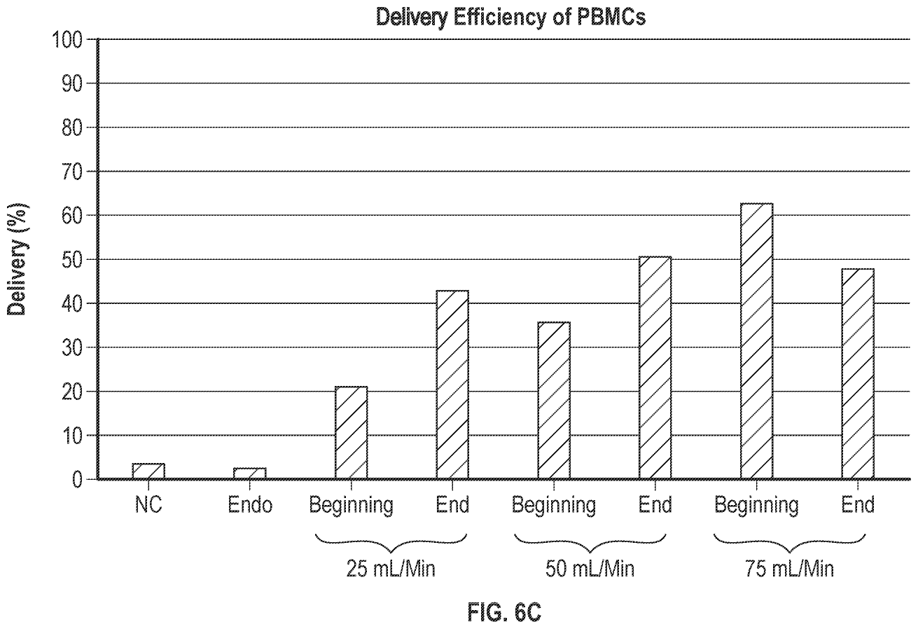

FIG. 6A illustrates the setup of the Example 3 tubing set. The cell perturbation filter used in Example 3 was a 13 mm² polymer filter (or membrane) having 5 μm pores. The setup includes a check valve (pressure regulator) fluidly connected to the cell mixture source (i.e., the cell processing system) that ensures the cell mixture is at a pressure adequate for perturbing cell membranes prior to passing through the tubing set. The setup also includes a (105 μm) or cell strainer upstream of the check valve for removing larger unwanted particulates in the cell mixture prior to passing the mixture through the cell perturbation filter. The setup of Example 3 also includes a pulsation damper for reducing any pressure oscillations introduced to the tubing set by the upstream cell processing system. The pulsation damper is tubing that is 300 mm in length and fluidly connects to the flow path of the tubing set via a y-connector. As noted above in Example 2, the pulsation damper may be optional in tubing sets and included based on the pressure oscillation profile introduced by the upstream cell processing system. As noted above in Example 1, the other illustrated components in the tubing set in FIG. 6A (e.g., pressure transducers, stopcock valves, and/or flow meters) are for testing the tubing set. One or more of these components may be eliminated from the tubing set when integrating it to a cell processing system for clinical/research use.

The samples tested included three samples that were passed through the tubing set at different rates—25 mL/min, 50 mL/min and 75 mL/min. Two control samples were also tested, including a no contact (“NC”) sample and endocytosis (“endo”) sample (each of which are described above in Example 1).

The results of testing the Example 3 tubing set (illustrated in FIG. 6A) integrated with the Lovo™ cell processing system are provided in Tables 3A-3B and FIGS. 6B-6C. The graphs illustrated in FIGS. 6B-6C correspond to the data presented in Table 3A. For each sample, the cell viability (Table 3A, FIG. 6B) and delivery efficiency (Table 3A, FIG. 6C) were measured at the beginning and at the end of collection. The estimated cell throughput was also measured and is provided below in Table 3B.

TABLE 3A
Viability and Delivery Efficiency of PBMCs

| | Viability | Delivery |
|---|---|---|
| Test | (%) | (%) |

| NC | | | 88.7 | 3.44 |
|---|---|---|---|---|
| Endo | | | 99 | 2.54 |
| | 25 | Beginning | 96.6 | 21 |
| | mL/min | End | 95.7 | 42.9 |
| | 50 | Beginning | 96.2 | 35.7 |
| | mL/min | End | 96.2 | 50.6 |
| | 75 | Beginning | 94.4 | 62.7 |
| | mL/min | End | 95.7 | 47.8 |

TABLE 3B
Estimated Throughput of PBMCs

| | Throughput |
|---|---|
| Test | (millions of cells) |
| 25 mL/min | 306 |
| 50 mL/min | 270 |
| 75 mL/min | 294 |

The cell viability of PBMCs passed through the Example 3 tubing set was above 90%, and above 95% in some instances, for each of the flow rates 25 mL/min, 50 mL/min, and 75 mL/min. Within each flow rate, there was less than about 1% of variance, and in some instances, no variance between the initial viability measurement and final viability measurement. The delivery efficiency of the Example 3 tubing set, quantified by measuring the delivery of the payload (Dextran) in the cells, generally increased as the flow rate was increased. For the 25 mL/min and 50 mL/min flow rates, the final delivery efficiency measured was greater than the initial delivery efficiency measured, with a difference of about 15-20% between the initial and final readouts in each flow rate. The final delivery efficiency was highest at the flow rate of 50 mL/min, about 50%. The final delivery efficiency was lowest at the flow rate of 25 mL/min, about 40%. The final delivery efficiency at the flow rate of 75 mL/min was between the two aforementioned flow rates at about 45%. The Example 3 tubing set was capable of processing over 250 million cells in one pass, and at a flow rate of 25 mL/min, over 300 million cells in one pass. As stated above, these results demonstrate the ability of the tubing sets described herein to process large quantities of cells.

### Example 4

Example 4 differs from the above Examples 1-3 because the cell type tested is activated T cells. The T cells were prepared (isolated and activated) using CD3/28 bead activation. After activation and isolation, the activated T cells were provided to a Fresenius Kabi Lovo™ cell processing system in which they were mixed with a payload (Dextran) to produce a cell mixture.

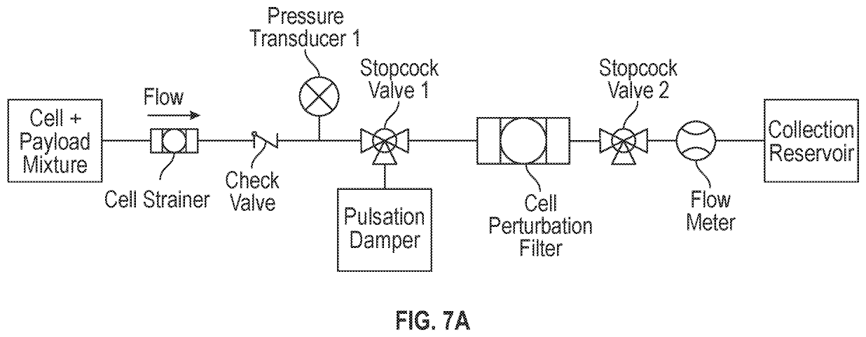

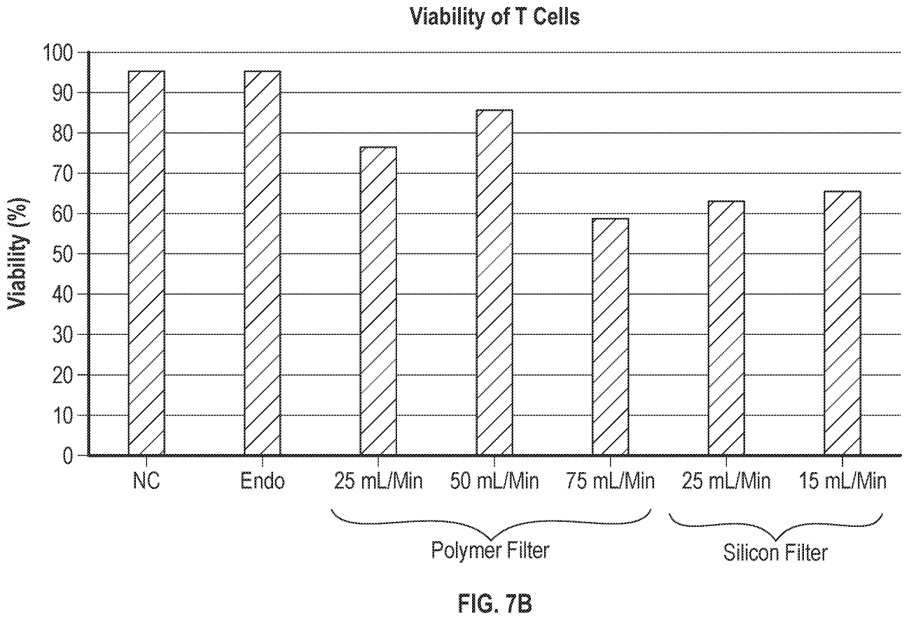

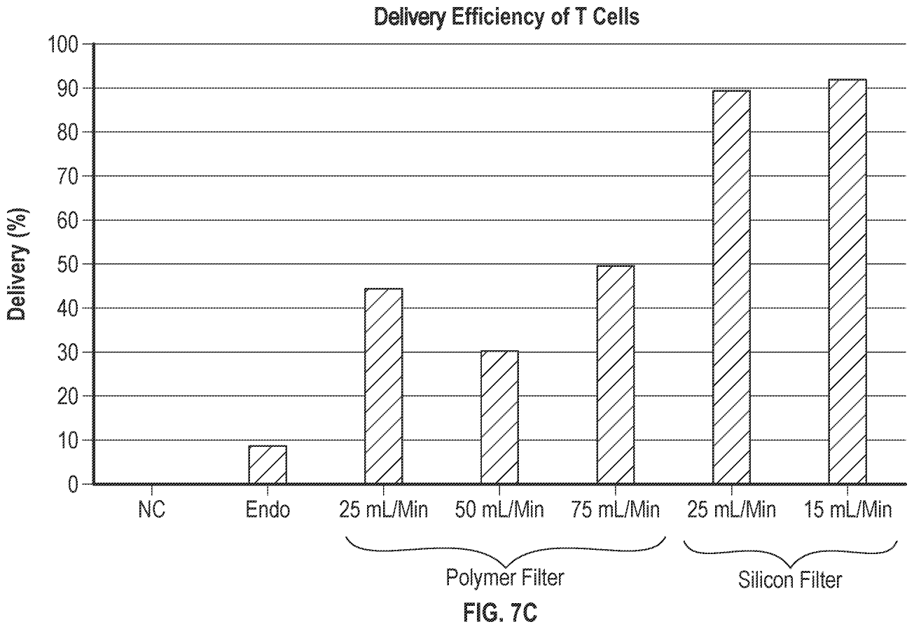

FIG. 7A illustrates the setup of the Example 4 tubing set. Two different types of cell perturbation filters were tested in Example 4—an 8 μm polymer filter (or membrane) and a 6 μm silicon filter (or membrane), the value referring to the pore size in the filter (e.g., 8 μm pore diameter and 6 μm pore diameter, respectively). The surface area of the polymer filter was 13 mm², and the surface area of the silicon filter was 7 mm². The Example 4 setup includes a check valve (pressure regulator) fluidly connected to the cell mixture source (i.e., the cell processing system) that, as stated above, ensures the cell mixture is at a pressure adequate for perturbing cell membranes prior to passing through the tubing set. The setup also includes a (105 μm) cell strainer upstream of the check valve for removing larger unwanted particulates in the cell mixture prior to passing the mixture through the cell perturbation filter. The setup of Example 4 also includes a pulsation damper that, as stated above, reduces any pressure oscillations introduced to the tubing set by the upstream cell processing system. The pulsation damper is tubing that is 300 mm in length and fluidly connects to the flow path of the tubing set via a stopcock valve. As noted above in Example 2, the pulsation damper may be optional in tubing sets and included based on the pressure oscillation profile introduced by the upstream cell processing system. As noted above in Example 1, the other illustrated components in the tubing set in FIG. 7A (e.g., pressure transducers, stopcock valves, and/or flow meters) are for testing the tubing set. One or more of these components may be eliminated from the tubing set when integrating it to a cell processing system for clinical/research use.

The samples tested included five samples across two filter types that were passed through the tubing set at different rates—15 mL/min, 25 mL/min, 50 mL/min and 75 mL/min. Two control samples were also tested, including a no contact (“NC”) sample and endocytosis (“endo”) sample (each of which are described above in Example 1).

The results of testing the Example 4 tubing set (illustrated in FIG. 7A) integrated with the Lovo™ cell processing system are provided in Tables 4A-4B and FIGS. 7B-7C. The graphs illustrated in FIGS. 7B-7C correspond to the data presented in Table 4A. For each sample, the cell viability (Table 4A, FIG. 7B) and delivery efficiency (Table 4A, FIG. 7C) were measured. The estimated cell throughput was also measured and is provided below in Table 4B.

TABLE 4A
Viability and Delivery Efficiency of Activated T Cells

| | Viability | Delivery |
|---|---|---|
| Test | (%) | (%) |

| NC | | | 95.4 | 0.13 |
|---|---|---|---|---|
| Endo | | | 95.3 | 8.5 |
| | Polymer | 25 mL/min | 76.6 | 44.2 |
| | Filter | 50 mL/min | 85.8 | 30 |
| | | 75 mL/min | 58.8 | 49.5 |
| | Silicon | 25 mL/min | 63 | 89.3 |
| | Filter | 15 mL/min | 65.5 | 91.9 |

TABLE 4B
Estimated Throughput of Activated T Cells

| | Throughput |
|---|---|
| Test | (millions of cells) |

| | | |
|---|---|---|
| Polymer | 25 mL/min | 221 |
| Filter | 50 mL/min | 319 |
| | 75 mL/min | 331 |
| Silicon | 25 mL/min | 282 |
| Filter | 15 mL/min | 257 |

The cell viability of activated T cells passed through the Example 4 tubing set was above 50% for each of the filter designs tested at varying flow rates between 15 mL/min and 75 mL/min. The highest cell viability of about 85% was observed at the flow rate of 50 mL/min using a polymer cell perturbation filter. The lowest cell viability of just below 60% was observed at the flow rate of 75 mL/min. At each flow rate of 15 mL/min and 25 mL/min, the cell viability using the silicon cell perturbation filter was greater than 60%. The delivery efficiency of the Example 4 tubing set, quantified by measuring the delivery of the payload (Dextran) in the cells, varied across different flow rates and filter designs. The delivery efficiency was highest using the silicon filter—at each of the tested flow rates of 15 mL/min and 50 mL/min, it was about 90%. The delivery efficiency was lowest at the flow rate of 50 mL/min (using the polymer filter), about 30%. The delivery efficiency at the other tested flow rates (25 mL/min and 75 mL/min) using the polymer filter was between about 45-50%. The Example 4 tubing set was capable of processing over 200 million cells in one pass, and in some instances, over 300 million cells in one pass, across the different flow rates and filter types, demonstrating the ability of the tubing sets described herein to process large quantities of cells.

### Example 5

Similar to the Example 4 above, the cell type tested in Example 5 was activated T cells. The T cells were prepared (isolated and activated) using CD3/28 bead activation. Example 5 differs from Example 4 because after activation and isolation, the activated T cells were provided to a Thermo Fisher Rotea™ cell processing system in which they were mixed with a payload (Dextran) to produce a cell mixture.

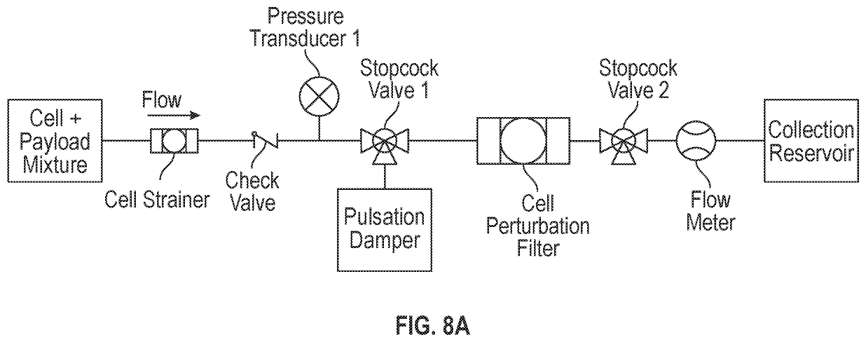

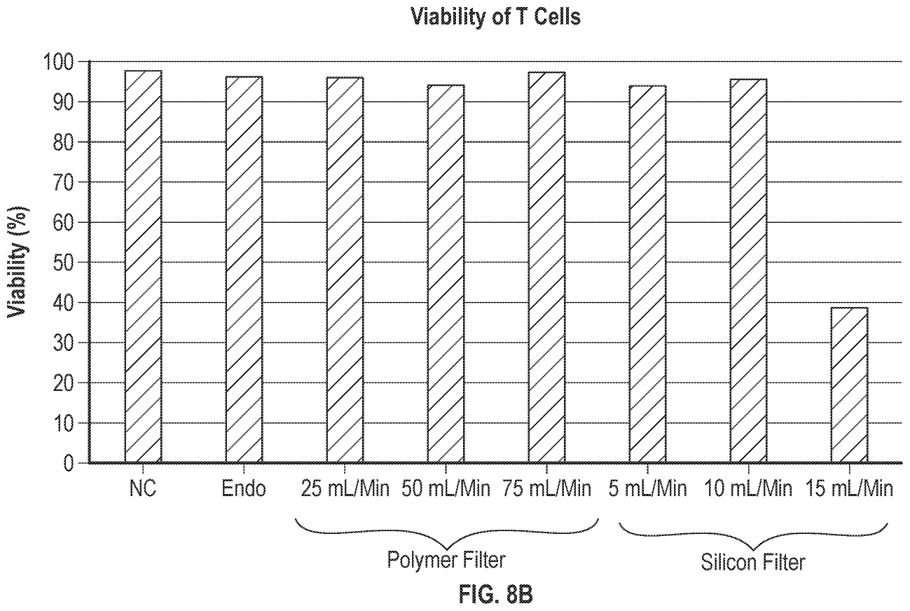

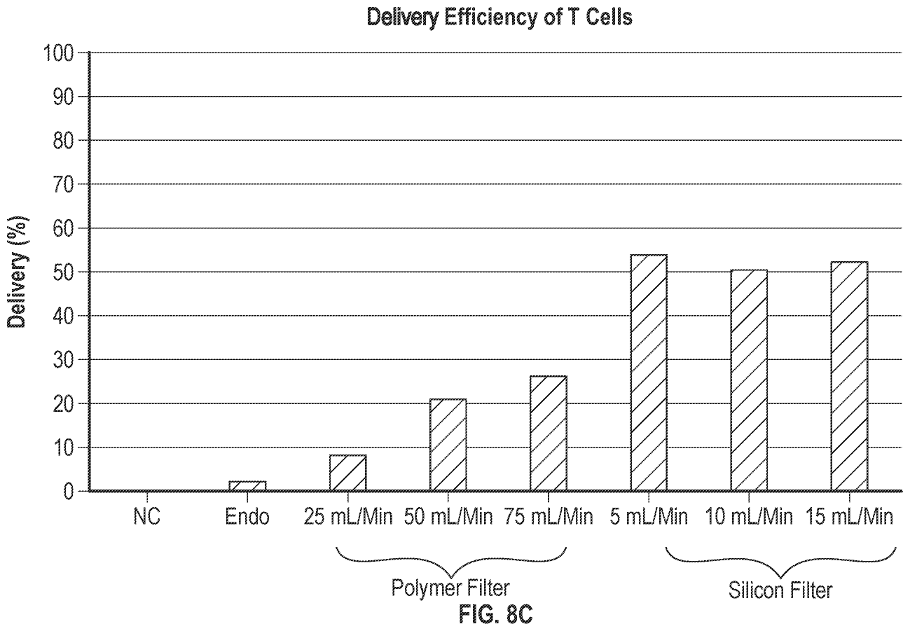

FIG. 8A illustrates the setup of the Example 5 tubing set. Two different types of cell perturbation filters were tested in Example 5—an 8 μm polymer filter (or membrane) and a 7 μm silicon filter (or membrane), the value referring to the pore size in the filter (e.g., 8 μm pores and 7 μm pores, respectively). The surface area of the polymer filter was 13 mm², and the surface area of the silicon filter was 7 mm². The Example 5 setup includes a check valve (pressure regulator) fluidly connected to the cell mixture source (i.e., the cell processing system). The setup also includes a (105 μm) cell strainer upstream of the check valve. The setup of Example 5 also includes a pulsation damper to reduce any pressure oscillations introduced to the tubing set by the upstream cell processing system. The pulsation damper is a column of tubing that is 300 mm in length and fluidly connects to the flow path of the tubing set via a stopcock valve. As noted above, the pulsation damper may be optional in tubing sets and included based on the pressure oscillation profile introduced by the upstream cell processing system. As noted above, the other illustrated components in the tubing set in FIG. 8A (e.g., pressure transducers, stopcock valves, and/or flow meters) are for testing the tubing set. One or more of these components may be eliminated from the tubing set when integrating it to a cell processing system for clinical/research use.

The samples tested included six samples across two filter types that were passed through the tubing set at six different rates—5 mL/min, 10 mL/min, 15 mL/min, 25 mL/min, 50 mL/min and 75 mL/min. Two control samples were also tested, including a no contact (“NC”) sample and endocytosis (“endo”) sample (each of which are described above in Example 1).

The results of testing the Example 5 tubing set (illustrated in FIG. 8A) integrated with the Rotea™ cell processing system are provided in Tables 5A-5B and FIGS. 8B-8C. The graphs illustrated in FIGS. 8B-8C correspond to the data presented in Table 5A. For each sample, the cell viability (Table 5A, FIG. 8B) and delivery efficiency (Table 5A, FIG. 8C) were measured. The estimated cell throughput was also measured and is provided below in Table 5B.

TABLE 5A
Viability and Delivery Efficiency of Activated T Cells

| | Viability | Delivery |
|---|---|---|
| Test | (%) | (%) |

| NC | | | | 97.9 | 0.018 |
|---|---|---|---|---|---|
| Endo | | | | 96.5 | 2.52 |
| | Polymer | 25 | mL/min | 96.1 | 8.41 |
| | Filter | 50 | mL/min | 94.4 | 21.1 |
| | | 75 | mL/min | 97.5 | 26.4 |
| | Silicon | 5 | mL/min | 94.1 | 54.1 |
| | Filter | 10 | mL/min | 95.6 | 50.5 |
| | | 15 | mL/min | 38.9 | 52.3 |

TABLE 5B
Estimated Throughput of Activated T Cells

| | | | Throughput |
|---|---|---|---|
| Test | | | (millions of cells) |
| Polymer | 25 | mL/min | 200 |
| Filter | 50 | mL/min | 217 |
| | 75 | mL/min | 225 |
| Silicon | 5 | mL/min | 198 |
| Filter | 10 | mL/min | 157 |

The cell viability of activated T cells passed through the Example 5 tubing set was above 90%, and in most cases, above 95% across the different filter designs and varying flow rates between 5 mL/min and 75 mL/min, disregarding the 15 mL/min flow rate using the silicon filter measurement. The delivery efficiency of the Example 5 tubing set, quantified by measuring the delivery of the payload (Dextran) in the cells, increased as the flow rate increased for the polymer filter samples, and was about the same across the silicon filter samples. The highest observed delivery efficiency was using the silicon filter—at each of the tested flow rates of 5 mL/min, 10 mL/min, and 15 mL/min, it was about 50%. The delivery efficiency was lowest at the flow rate of 25 mL/min (using the polymer filter), about 10%. The delivery efficiency at the other tested flow rates (50 mL/min and 75 mL/min) using the polymer filter was between about 20-25%. The Example 5 tubing set was capable of processing over 150 million cells in one pass, and in some instances, over 200 million cells in one pass, across the different flow rates and filter types, demonstrating the ability of the tubing sets described herein to process large quantities of cells.

### Example 6

Similar to Examples 1-3 above, the cell type tested in Example 6 was PBMCs. The PBMCs were prepared using Ficoll separation. After isolation, the PBMCs were provided to a Fresenius Kabi Lovo™ cell processing system in which they were mixed with a payload (Dextran) to produce a cell mixture.

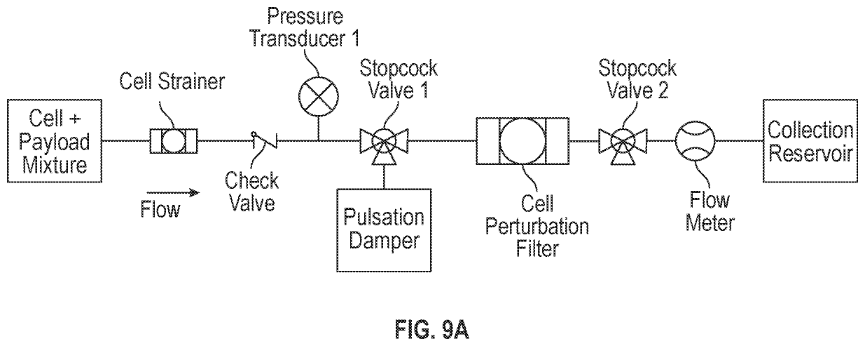

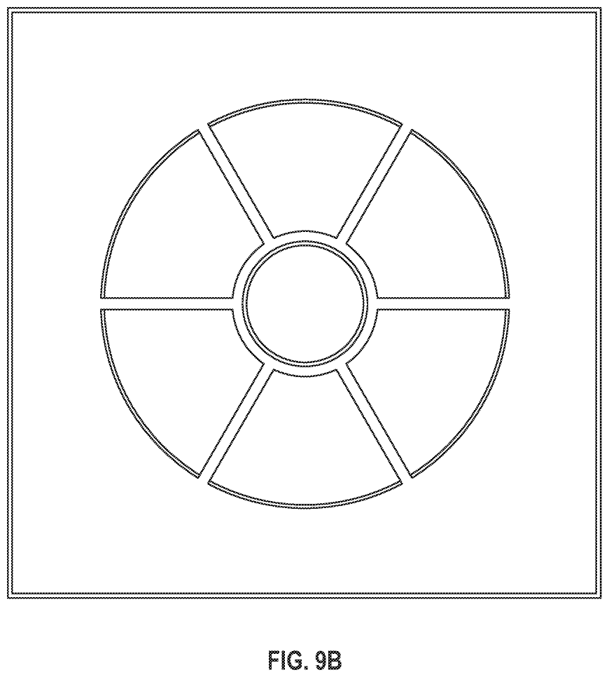

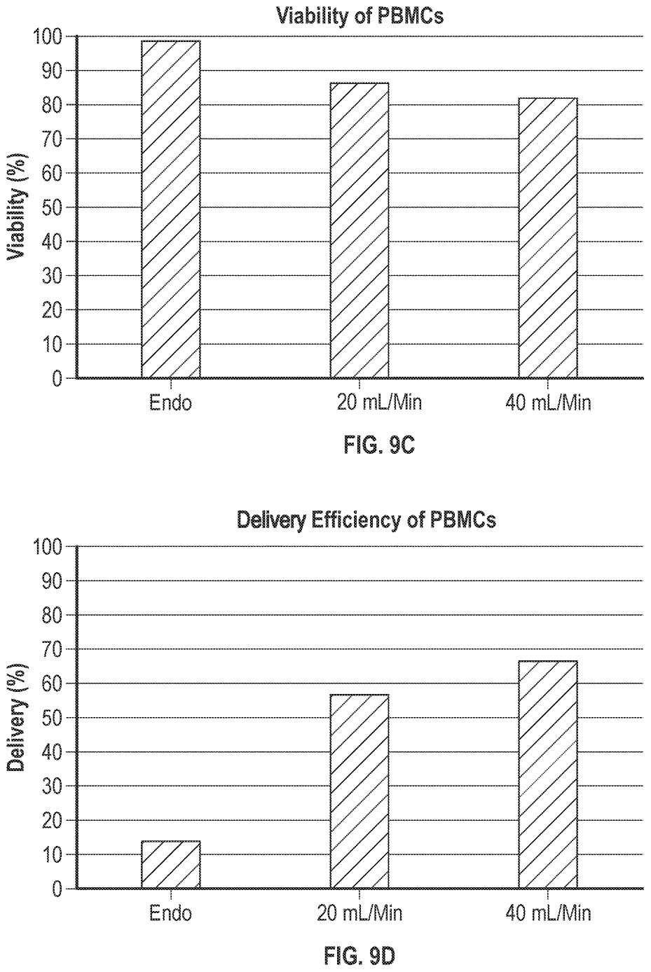

FIG. 9A illustrates the setup of the Example 6 tubing set. The cell perturbation filter tested in Example 6 was a 5 μm silicon filter (or membrane), the design of which is illustrated in FIG. 9B. 5 μm refers to the diameter of the pores in the filter. The surface area of the silicon filter was 7 mm². As shown in FIG. 9B, the silicon filter tested in Example 6 includes a support structure that enables the silicon filtering surface to withstand high pressures and/or flow rates. The Example 6 setup includes a check valve (pressure regulator) fluidly connected to the cell mixture source (i.e., the cell processing system). The setup also includes a (105 μm) cell strainer upstream of the check valve. The setup of Example 6 also includes a pulsation damper to reduce any pressure oscillations introduced to the tubing set by the upstream cell processing system. The pulsation damper is tubing that is 300 mm in length and fluidly connects to the flow path of the tubing set via a stopcock valve. As noted above, the pulsation damper may be optional in tubing sets and included based on the pressure oscillation profile introduced by the upstream cell processing system. As noted above, the other illustrated components in the tubing set in FIG. 9A (e.g., pressure transducers, stopcock valves, and/or flow meters) are for testing the tubing set. One or more of these components may be eliminated from the tubing set when integrating it to a cell processing system for clinical/research use.

The samples tested included two samples that were passed through the tubing set at different rates—20 mL/min, and 40 mL/min. A control endocytosis (“endo”) sample was also tested (described above in Example 1).

The results of testing the Example 6 tubing set (illustrated in FIG. 9A) integrated with the Lovo™ cell processing system are provided in Tables 6A-6B and FIGS. 9C-9D. The graphs illustrated in FIGS. 9C-9D correspond to the data presented in Table 6A. For each sample, the cell viability (Table 6A, FIG. 9C) and delivery efficiency (Table 6A, FIG. 9D) were measured. The estimated cell throughput was also measured and is provided below in Table 6B.

TABLE 6A
Viability and Delivery Efficiency of PBMCs

| | Viability | Delivery |
|---|---|---|
| Test | (%) | (%) |
| Endo | 98.6 | 14.1 |
| 20 mL/min | 86.6 | 56.7 |
| 40 mL/min | 82.1 | 66.8 |

TABLE 6B
Estimated Throughput of PBMCs

| | Throughput |
|---|---|
| Test | (millions of cells) |
| 20 mL/min | 647 |
| 40 mL/min | 529 |

The cell viability of PBMCs passed through the Example 6 tubing set was above 80% at each of the flow rates 20 mL/min and 40 mL/min. The cell viability was slightly higher for the 20 mL/min flow rate at above about 85%. The delivery efficiency of the Example 6 tubing set, quantified by measuring the delivery of the payload (Dextran) in the cells, increased from the 20 mL/min flow rate to the 40 mL/min flow rate, and was above 50% for both flow rates. The highest delivery efficiency measured was at the flow rate of 40 mL/min, about 65%. The Example 6 tubing set was capable of processing over 500 million cells in one pass, and at the flow rate of 20 mL/min, almost 600 million cells in one pass. The large quantity of cells passed through the Example 6 tubing set may be in part due to the design of the otherwise delicate silicon filter (e.g., the support structure on the filtering surface). In any instance, the cell throughput measured for the Example 6 tubing set, similar to the above Examples, demonstrates the ability of the tubing sets described herein to process large quantities of cells.

### Example 7

To perform the experiment, a Leukopak sample was retrieved, and T cells were isolated using a Pan-T Cell Isolation Kit and freeze-preserved in liquid nitrogen. The isolated T cells were activated with TransAct and cultured in G-Rex multi-well plates for 10 days, with 10 million cells per well. After isolation, the activated T cells were provided to a Fresenius Kabi Lovo™ cell processing system in which they were mixed with a payload (Dextran) to produce a cell mixture. The setup of the tubing kit integrated with the cell processing system was as illustrated in FIG. 1E (described above), including a cell strainer, a check valve (pressure regulator), and pulsation damper (in that order) fluidly connected between the cell processing system and the cell perturbation filter. The pulsation damper was a column of tubing having one end closed and fluidly connected to the primary flow line through the tubing kit via a y-connector.

Two different silicon filter sizes were tested in the Example 7 tubing set—a 5 μm silicon filter and a 6 μm silicon filter, the value (5 μm and 6 μm) referring to the diameter of each of the pores in the filter. The surface area of each of the silicon filters was 7 mm². The design of each of the filters was as illustrated in FIG. 9B described above-they included support structures that enables the filters to withstand high pressures and/or flow rates. The 5 μm silicon filter design was tested at a flow rate of 40 mL/min. The 6 μm silicon filter design was tested at flow rates of 40 mL/min and 60 mL/min. Two control samples were also tested, including a no contact (“NC”) sample and endocytosis (“endo”) sample (each of which are described above in Example 1).

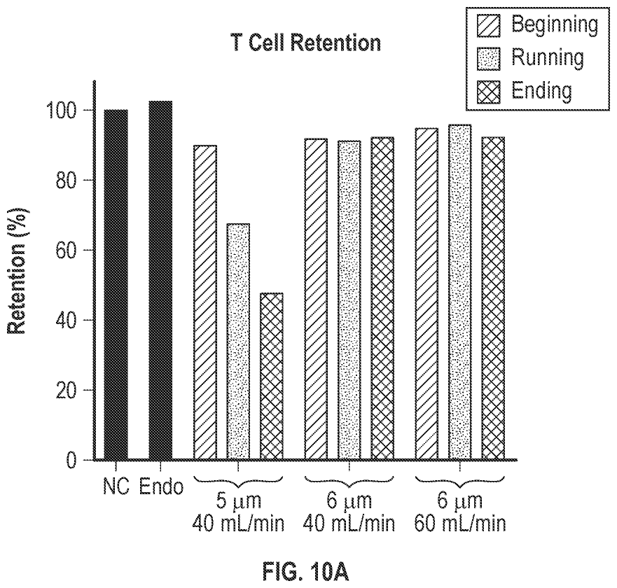

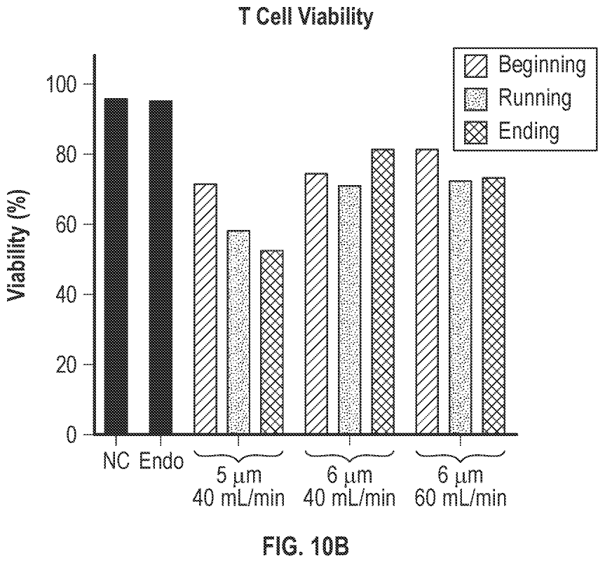

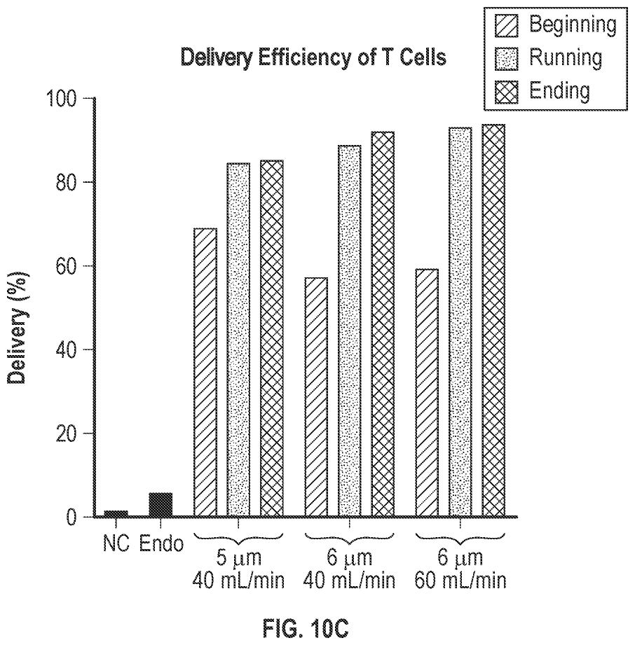

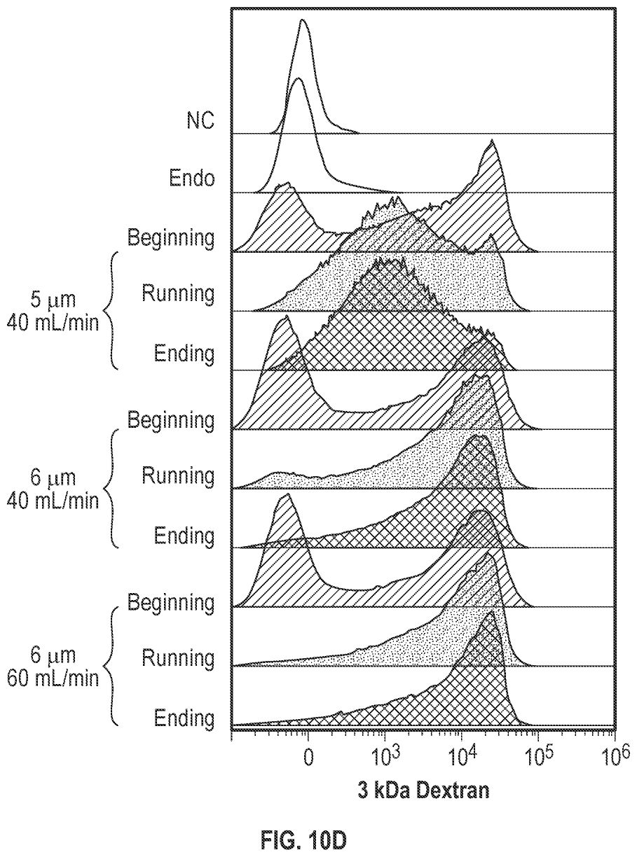

The results of testing the Example 7 tubing set integrated with the Lovo™ cell processing system are provided in FIGS. 10A-10D. For each sample, the cell retention (FIG. 10A), cell viability (FIG. 10B), and delivery efficiency (FIGS. 10C and 10D) were measured at the beginning, middle (“running”), and end of collection.

Cell retention is a measure of the quantity of cells in the cell mixture that are retained when the cell mixture is passed through the tubing set from the Lovo™ cell processing system. As shown in FIG. 10A, the cell retention of activated T cells measured for both the 40 mL/min and 60 mL/min flow rates using the 6 μm silicon filter was high at the end of collection, above about 90%, indicating that most of the cells in the mixture were retained when passed through the Example 7 tubing set. Notably, the cell retention of activated T cells measured at the beginning, middle, and end of collection for both the 40 mL/min and 60 mL/min flow rates using the 6 μm silicon filter exhibited minimal variability, less than about 5%. The 5 μm filter tested at the 40 mL/min flow rate demonstrated the lowest cell retention at about 50% measured at the end of collection, a decrease of about 40% from the cell retention measured at the beginning of collection for the sample. This measured cell retention indicated that a 5 μm pore size may be too small for efficacious mechanoporation of activated T cells.

The cell viability of T cells passed through the Example 7 tubing set having the 6 μm silicon filter and at each of the flow rates of 40 mL/min and 60 mL/min is above 70%. The highest measured cell viability at the end of collection was measured when using the 6 μm silicon filter at a flow rate of 40 mL/min, about 80%. An overall increase in measured cell viability was observed for the 6 μm, 40 mL/min sample, about 5% between the beginning and the end of collection. A decrease in measured cell viability was observed for each of the 5 μm, 40 mL/min and 6 μm, 60 mL/min samples. The cell viability measured at the end of collection for the 6 μm, 60 mL/min sample was about 70% (about 10% lower than the cell viability measured at the beginning of collection). The cell viability of T cells passed through the Example 7 tubing set having the 5 μm silicon filter and at a flow rate of 40 mL/min was above 50% at the end of collection. The cell viability decreased from about 70% at the beginning of collection, to about 60% at the middle of collection, and to about 50% at the end of collection.

The delivery efficiency of the Example 7 tubing set, quantified by measuring the delivery of the payload (Dextran) in the cells (FIG. 10C) as well as the presence of Dextran in the T cells (FIG. 10D), demonstrates high delivery efficiency of the payload into the cells. In each of the samples, the measured delivery efficiency increased from the beginning of collection to the end of collection. At the end of collection, the delivery efficiency of each of the samples was above about 80%. For the 5 μm, 40 mL/min sample, the measured delivery efficiency at the end of collection was about 82% (about 15-20% greater than the initial measured delivery efficiency). For each of the 40 mL/min and 60 mL/min flow rates tested with the 6 μm silicon filter, the final delivery efficiency measured was about 90% (greater than the initial delivery efficiency measured for each sample by about 30%). In each of the three samples, the delivery efficiency measured during collection (“running”) and at the end of collection varied minimally, with less than about 5% of variability.

What is claimed is:

1. A tubing set for delivering a payload into cells, the tubing set comprising:
a cell source tubing portion configured to fluidly connect to a cell source comprising a cell suspension;
a mixer fluidly connected to the cell source tubing portion and configured to mix the payload and the cell suspension to produce a mixture;
a delivery portion comprising one or more cell perturbation filters fluidly connected to the mixer to receive the mixture and configured to perturb membranes of cells of the mixture as the mixture passes through the one or more cell perturbation filters; and
a pump connection portion configured to connect to one or more pumps that are configured to move the cell suspension through the cell source tubing portion and into the mixer, to move the mixture from the mixer through the one or more cell perturbation filters, and to move a perturbed cell mixture into one or more collection reservoirs configured to collect the perturbed cell mixture.

2. The tubing set of claim 1, comprising a payload source tubing portion fluidly connected to the mixer and configured to fluidly connect to a payload source comprising the payload.

3. The tubing set of claim 2, wherein the one or more pumps are configured to move the payload through the payload source tubing portion and into the mixer.

4. The tubing set of claim 2 or claim 3, wherein the cell source tubing portion comprises a cell suspension pressure regulator and the payload source tubing portion comprises a payload pressure regulator.

5. The tubing set of claim 4, wherein one or more of the cell suspension pressure regulator or the payload pressure regulator comprises a check valve.

6. The tubing set of any one of claims 2-5, wherein the payload source is fluidly connected to the cell source to receive the cell suspension from the cell source.

7. The tubing set of claim 6, comprising a single pressure regulator fluidly connected to the payload source to receive the cell suspension and the payload.

8. The tubing set of any one of claims 1-7, wherein the mixer is configured to normalize pulsations from the one or more pumps.

9. The tubing set of any one of claims 1-8, comprising a mixture pressure regulator fluidly connected to the mixer to receive the mixture.

10. The tubing set of any one of claims 1-9, wherein the mixer comprises an intersecting flow path, in-line agitating geometry, or a Venturi mixer in the tubing set.

11. The tubing set of any one of claims 1-10, wherein the mixer is configured to mix the payload and the cell suspension without an agitation device or impeller.

12. The tubing set of any one of claims 1-11, wherein each filter of the one or more cell perturbation filters comprises a porous surface, each pore of the porous surface comprising a diameter between 1 μm and 20 μm.

13. The tubing set of claim 12, wherein the porous surface comprises uniformly-sized pores.

14. The tubing set of claim 12 or claim 13, wherein the diameter of the pores is between 10% and 90% of a diameter of the cells.

15. The tubing set of any one of claims 1-14, wherein the one or more cell perturbation filters comprises a thickness between 0.1 μm and 1 mm.

16. The tubing set of any one of claims 1-15, wherein the one or more cell perturbation filters comprises a plurality of cell perturbation filters.

17. The tubing set of claim 16, wherein the plurality of cell perturbation filters are disposed in the tubing set in parallel to one another, such that each filter of the plurality of cell perturbation filters is fluidly connected to the mixer and is configured to be fluidly connected to the one or more collection reservoirs.

18. The tubing set of claim 17, wherein the one or more collection reservoirs comprises a single collection reservoir.

19. The tubing set of claim 17 or claim 18, wherein the one or more collection reservoirs comprises a plurality of collection reservoirs, each collection reservoir of the plurality of collection reservoirs configured to be fluidly connected to a given filter of the plurality of cell perturbation filters.

20. The tubing set of any one of claims 1-19, wherein the one or more cell perturbation filters comprises silicon nitride, polycarbonate, or another polymer.

21. The tubing set of any one of claims 1-20, comprising one or more leukocyte reduction filters or cell strainers.

22. The tubing set of claim 21, wherein the one or more leukocyte reduction filters or cell strainers are fluidly connected to the cell source to receive the cell suspension.

23. The tubing set of claim 21 or claim 22, wherein the cell source tubing portion comprises the one or more leukocyte reduction filters or cell strainers.

24. The tubing set of any one of claims 1-23, comprising a collection reservoir tubing portion fluidly connected to the delivery portion and configured to fluidly connect to the one or more collection reservoirs.

25. The tubing set of any one of claims 1-24, comprising one or more of polyvinyl chloride tubing or C-flex tubing.

26. The tubing set of any one of claims 1-25, comprising a housing containing at least the mixer and the one or more cell perturbation filters.

27. The tubing set of claim 26, wherein the housing is configured to be removably connected to the tubing set.

28. The tubing set of claim 26 or claim 27, wherein the housing contains the one or more collection reservoirs.

29. The tubing set of any one of claims 26-28, wherein the housing comprises a payload reservoir configured to contain the payload.

30. The tubing set of claim 29, wherein the tubing set is provided with the payload stored in the payload reservoir.

31. The tubing set of claim 29 or claim 30, wherein the payload reservoir is configured to normalize pulsations from the one or more pumps.

32. The tubing set of any one of claims 1-31, wherein the mixer is configured to contain the payload.

33. The tubing set of claim 32, wherein the tubing set is provided with the payload stored in the mixer.

34. The tubing set of claim 32 or claim 33, wherein the mixer is configured to normalize pulsations from the one or more pumps.

35. The tubing set of any one of claims 1-34, wherein the delivery portion is configured to receive a volume between at least 1 mL and 500 mL.

36. The tubing set of any one of claims 1-35, wherein the delivery portion is configured to receive a volume between at most 500 mL and 5 L.

37. The tubing set of any one of claims 1-36, wherein the one or more pumps comprises a single pump.

38. The tubing set of claim 37, wherein the pump comprises a peristaltic pump or syringe pump.

39. The tubing set of any one of claims 1-38, comprising a pulsation damper configured to normalize pulsations from the one or more pumps.

40. The tubing set of claim 39, wherein the pulsation damper is fluidly connected to the mixer to receive the mixture.

41. The tubing set of any one of claims 1-40, wherein the tubing set is configured to fluidly connect to one or more of the cell source, a payload source, and the one or more collection reservoirs using tube welded or swaged connections.

42. The tubing set of any one of claims 1-41, wherein the tubing set is configured to fluidly connect to one or more of the cell source, a payload source, and the one or more collection reservoirs using one or more connectors.

43. The tubing set of claim 42, wherein the one or more connectors comprise one or more Luer connectors, threaded connectors, or quick disconnect connectors.

44. The tubing set of any one of claims 1-43, wherein the tubing set does not comprise flow sensors or pressure sensors.

45. A mechanoporation system for delivering a payload into cells, the system comprising:
a payload source comprising the payload;
a cell source comprising a cell suspension;
a tubing set comprising:
a mixer fluidly connected to the payload source and to the cell source and configured to mix the payload and the cell suspension to produce a mixture; and
one or more cell perturbation filters fluidly connected to the mixer to receive the mixture and configured to perturb membranes of cells of the mixture as the mixture passes through the one or more cell perturbation filters;
one or more collection reservoirs fluidly connected to the one or more cell perturbation filters to collect the perturbed cell mixture; and
one or more pumps connected to the tubing set and configured to move the payload and the cell suspension into the mixer, to move the mixture from the mixer through the one or more cell perturbation filters, and to move the perturbed cell mixture into the one or more collection reservoirs.

46. The system of claim 45, wherein the payload source comprises a flexible bag or vial comprising the payload.

47. The system of claim 45 or claim 46, wherein the cell source comprises a flexible bag or vial comprising the cell suspension.

48. A method for delivering a payload into cells, the method comprising:
mixing, by a mixer fluidly connected to a payload source comprising a payload and to a cell source comprising a cell suspension, the payload and the cell suspension to produce a mixture;
perturbing, by one or more cell perturbation filters fluidly connected to the mixer to receive the mixture, membranes of cells of the mixture as the mixture passes through the one or more cell perturbation filters; and
collecting, by one or more collection reservoirs fluidly connected to the one or more cell perturbation filters, the perturbed cell mixture,
wherein one or more pumps are configured to move the payload and the cell suspension into the mixer, to move the mixture from the mixer through the one or more cell perturbation filters, and to move the perturbed cell mixture into the one or more collection reservoirs.

49. The method of claim 48, wherein a speed of a cell in the cells of the mixture as the mixture passes through the one or more cell perturbation filters is between 10 mm/s and 100 m/s.

50. The tubing set of any one of claims 1-44, wherein the tubing set is configured to provide the perturbed cell mixture having a cell viability of at least 60%.

51. The tubing set of any one of claim 1-44 or 50, wherein a delivery efficiency of the tubing set in perturbing the membranes of the cells is at least 60%.

52. The tubing set of any one of claim 1-44, 50, or 51, wherein the one or more cell perturbation filters comprises silicon, silicon nitride, silicon oxide, polycarbonate, or another polymer.

53. The tubing set of any one of claim 1-44 or 50-52, wherein the mixer comprises a portion of tubing configured to receive and subsequently release at least a portion of the cell suspension and the payload to normalize pulsations from the one or more pumps.
